SEQUENCE LISTING (3 sequences)

SEQ ID NO: 1 - protein, 288 aa, Homo sapiens
MKDRTQELRTAKDSDDDDDVAVTVDRDRFMDEFFEQVEEIRGFIDKIAENVEEVKRKHSAILASPNPDEKTKEELEELMSDIKKTANKVRSKLKSIEQSIEQEEGLNRSSADLRIRKTQHSTLSRKFVEVMSEYNATQSDYRERCKGRIQRQLEITGRTTTSEELEDMLESGNPAIFASGIIMDSSISKQALSEIETRHSEIIKLENSIRELHDMEMDMAMLVESQGEMIDRIEYNVEHAVDYVERAVSDTKKAVKYQSKARRKKIMIIICCVILGIVIASTVGGIFA

SEQ ID NO: 2 - protein, 290 aa, Artificial Sequence
  Description of Artificial Sequence  Synthetic Peptide
MSYNNPYQLETPFEESYELDEGSSAIGAEGHDFVGFMNKISQINRDLDKYDHTINQVDSLHKRLLTEVNEEQASHLRHSLDNFVAQATDLQFKLKNEIKSAQRDGIHDTNKQAQAENSRQRFLKLIQDYRIVDSNYKEENKEQAKRQYMIIQPEATEDEVEAAISDVGGQQIFSQALLNANRRGEAKTALAEVQARHQELLKLEKSMAELTQLFNDMEELVIEQQENVDVIDKNVEDAQLDVEQGVGHTDKAVKSARKARKNKIRCWLIVFAIIVVVVVVVVVPAVVKTR

SEQ ID NO: 3 - protein, 295 aa, Artificial Sequence
  Description of Artificial Sequence  Synthetic Peptide
MSNANPYENNNPYAENYEMQEDLNNAPTGHSDGSDDFVAFMNKINSINANLSRYENIINQIDAQHKDLLTQVSEEQEMELRRSLDDYISQATDLQYQLKADIKDAQRDGLHDSNKQAQAENCRQKFLKLIQDYRIIDSNYKEESKEQAKRQYTIIQPEATDEEVEAAINDVNGQQIFSQALLNANRRGEAKTALAEVQARHQELLKLEKTMAELTQLFNDMEELVIEQQENVDVIDKNVEDAQQDVEQGVGHTNKAVKSARKARKNKIRCLIICFIIFAIVVVVVVVPSVVETRK

(19) United States
(12) Patent Application Publication
(10) Pub. No.: US 2005/0136394 A1
(43) Pub. Date: Jun. 23, 2005

(54) Cell-based assay for identifying peptidase inhibitors

(76) Inventors: Hong Fang, Chapmansboro, TN (US); Neil Green, Chapmansboro, TN (US)

Correspondence Address: FULBRIGHT & JAWORSKI L.L.P.;SUITE 2400, 600 CONGRESS AVENUE, AUSTIN, TX, 78701-3271, US

(21) Appl. No.: 10/842,846

(22) Filed: May 11, 2004

Related U.S. Application Data
(60) Provisional application No. 60/480,625, filed on Jun. 23, 2003.

(51) Int. Cl. C12Q 1/00; C12Q 1/37

(52) U.S. Cl. 435/4; 435/23

(57) ABSTRACT

The present invention provides assays for the identification of inhibitors of endopeptidase toxins. The assays utilize genetically engineered yeast cells that contain a conditionally expressed endopeptidase toxin. When conditions for expression of the toxin are met, the toxin cleaves a yeast (natural or engineered) peptide product that is required for yeast survival. If the yeast is grown in the presence of an candidate substance that is an inhibitor of the toxin, the yeast survives, thereby providing a rapid and sensitive identification of the inhibitor.

The present application claims benefit of priority to U.S. Provisional Ser. No. 60/480,625, filed Jun. 23, 2003, the entire contents of which are hereby incorporated by reference.

The government owns rights in the present invention pursuant to NSF CAREER Award #9985479 and NSF MCB #9604669, both from the National Science Foundation.

### BACKGROUND OF THE INVENTION

1. Field of the Invention

The invention relates generally to the fields of microbiology and pathology. More particularly, the invention relates to assays for the rapid identification of peptidase inhibitors.

2. Description of Related Art

The emerging bioterrorism threat has galvanized the need for rapidly effective treatments against deadly bacterial toxins. Many of the bacterial toxins are endopeptidases that destroy specific essential proteins within host cells. These bacterial toxins include, but are not limited to, botulinum neurotoxin (BONT) and anthrax lethal factor.

A traditional method of treating infections of this nature is by administering antibiotics. One of the major impediments in treating bacterial infections in this way is the limited susceptibility of bacteria to various antibiotics. Also, as bacteria reproduce quickly, any that are resistant to the drug used may quickly replace the ones that are killed, thereby further reducing the effectiveness of the drug.

Even with effective antibiotic control of infection through antibiotics, the effects of the toxins that have already been produced are not mitigated. Therefore, to counteract the effects of the toxins, another drug or treatment needs to be administered. One such class of drugs are bacterial endopeptidase inhibitors, which prevent the toxins from damaging host cells. However, identifying inhibitors of specific toxins is a time consuming process, requiring many tests and trials before a drug may be produced. For example, certain assays measure of peptidase activity using electrophoretic separation of the cleavage products—a slow and cumbersome approach. Assays using fluorogenic substrates have been developed, and liquid chromatography (HPLC) and mass spectroscopy provide promising new avenues of attack, but to date, these have not provided entirely satisfactory results. Thus, there remains a need for simple and fast methods of identifying and isolating bacterial endopeptidase inhibitors.

### SUMMARY OF THE INVENTION

Thus, in accordance with the present invention, there is provided a method of identifying an endopeptidase inhibitor comprising (a) providing a yeast cell, wherein said cell expresses a polypeptide that is essential to yeast cell viability or growth, wherein said polypeptide comprises or has been modified to comprise a cleavage site for said endopeptidase; (b) contacting said yeast cell and said endopeptidase in the presence of a candidate substance; and (c) assessing the viability and/or growth of said yeast cell, wherein improved viability and/or growth of said yeast cell in the presence of said candidate substance, as compared to viability and/or growth of said yeast cell in the absence of said candidate substance, identifies said candidate substance as a endopeptidase inhibitor. The endopeptidase may be a serine endopeptidase, a cysteine endopeptidase, an aspartic endopeptidase or a metallo endopeptidase, more particularly a bacterial toxin endopeptidase, and more specifically a Botulinum neurotoxin (BoNT), wherein said endopeptidase cleavage site is Q/F for BoNTB/LC, and K/A for BoNTC/LC. The modified polypeptide may also comprise a protease binding site.

The essential polypeptide may be Snc1 or Snc2 or Sso1 or Sso2. Viability may be measured by standard culture methods, by flow cytometry by selective staining, by the slide viability method, by flocculation test, or by fermentation test. Growth may be measured by assessing incorporation of radioactive nucleotides or by cell counting. The yeast cell may further comprises a null mutation in a functionally redundant homolog of said essential polypeptide. The yeast cell may also further comprise an endopeptidase transgene under the control of an inducible promoter, and contacting comprises growing said yeast cell under conditions that induce said promoter, thereby permitting expression of said endopeptidase in said yeast cell. The inducible promoter may be a yeast inducible promoter (e.g., Gal1, Gal10, GalS, or GalL, and said conditions that induce said promoter comprises culturing said yeast in galactose), or a non-yeast inducible promoter (e.g., a tetracycline-responsive promoter and said conditions that induce said promoter comprises culturing said yeast in tetracycline).

The candidate substance may be a peptide or polypeptide and providing said peptide or polypeptide comprises contacting said yeast cell with an expression construct encoding said peptide or polypeptide. The polypeptide may be an antibody or an enzyme. The candidate substance may be an organopharmaceutical or a siRNA.

In another embodiment, there is provided a yeast cell that expresses a polypeptide that is essential to yeast cell viability or growth, wherein said polypeptide comprises a heterologous cleavage site for a endopeptidase, and optionally a heterologous endopeptidase binding site. The yeast cell may further comprise a transgene encoding said endopeptidase under the control of an inducible promoter. The inducible promoter is a yeast inducible promoter or a non-yeast inducible promoter. The yeast cell may further comprise a null mutation in a functionally redundant homolog of said polypeptide that comprises said heterologous cleavage site.

The present invention, in all of the preceding embodiments, also envisions the use of exopeptidases in analogous assays. Such exopeptidases may have either C-terminal or N-terminal peptidase funtions.

It is contemplated that any method or composition described herein can be implemented with respect to any other method or composition described herein. The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Example of yeast cell-based assays using Snc2. The agar plate on the left contains glucose, and the agar plate on the right contains galactose.

FIG. 2—Example of yeast cell-based assays using Sso1/2. The agar plate on the left contains glucose, and the agar plate on the right contains galactose.

FIG. 3—Sequence alignments for Syntaxin and Sso1/2p. Shaded regions show areas of homology (identity or conservative substitution); syntaxin cleavage site indicated by an arrow.

### DESCRIPTION OF ILLUSTRATIVE EMBODIMENTS

I. The Present Invention

The present invention provides a rapid and sensitive system for identifying and isolating pharmaceutically effective compounds that inhibit the proteolytic activity of peptidases (also known as proteases), such as endo- and exopeptidases (endo- and exoproteases) within eukaryotic cells. In a particular embodiment, the assay of the invention makes use of recombinant yeast cells that harbor an endopeptidase that cleaves an essential yeast protein. In certain cases, the yeast cells will have been further engineered to comprise an essential protein that contains a heterologous proteolytic cleavage site for the endopeptidase in question. When expression of endopeptidase is induced, cleavage of the essential protein occurs and cell death ensues. However, inclusion of an appropriate endopeptidase inhibitor in this culture can block cleavage and prevent cell death.

This format is readily scalable so that one can screen large numbers of putative inhibitors, such as peptides, siRNA, antisense molecules, small molecules, in a rapid fashion. The assay offers several distinct advantages: (1) positive growth selection is a much more powerful, efficient and economic approach than existing screening procedures; (2) the technology employs function-based assays to isolate toxin inhibitors, which is preferable over the affinity binding-based assays mostly commonly used in inhibitor screening procedures; and (3) a one step cell-based assay not only selects for toxin inhibitors, but eliminates inhibitors that are toxic to yeast, a model eukaryotic cell that is related to human cells. Together, these advantages provide a faster screen for large numbers of candidate substances which are more likely to be effective and safe when applied to animals and humans. The details of this invention are described further in the following pages.

II. Peptidases, Endopeptidases and Exopeptidases

A peptidase is an enzyme that cleaves a peptide bond. An endopeptidase is any peptidase that catalyzes the cleavage of internal peptide bonds in a polypeptide or protein. Endopeptidases are divided into subclasses on the basis of catalytic mechanism: the serine endopeptidases, cysteine endopeptidases, aspartic endopeptidases, metalloendopeptidases, and other endopeptidases. Exopeptidases cleave proteins near the carboxy- or amino-termini, and thus are termed carboxy- or amino-exopeptidases.

There are a substantial number of different peptidases present in cells with differing specificities, so as to require different sequences and/or conformations of the polypeptide as the cleavage site. With hybrid DNA technology, one tries to provide a high level of production of a polypeptide product, which is in addition to the normal cellular products. Where such polypeptide requires processing, the cell may not be able to respond to the increased processing load. However, the mere fact of providing for enhanced genetic capability of producing the peptidase is no assurance that there will be an enhanced or more efficient processing of the peptidase substrate. See U.S. Pat. No. 5,077,204, herein incorporated by reference in its entirety.

A. Serine Endopeptidases

This class comprises two distinct families. The chymotrypsin family which includes the mammalian enzymes such as chymotrypsin, trypsin or elastase or kallikrein and the substilisin family which include the bacterial enzymes such as subtilisin. The general 3D structure is different in the two families but they have the same active site geometry and then catalysis proceeds via the same mechanism. The serine endopeptidases exhibit different substrate specificities which are related to amino acid substitutions in the various enzyme subsites interacting with the substrate residues. Some enzymes have an extended interaction site with the substrate whereas others have a specificity restricted to the P1 substrate residue.

Three residues which form the catalytic triad are essential in the catalytic process, i.e., His 57, Asp 102 and Ser 195 (chymotrypsinogen numbering). The first step in the catalysis is the formation of an acyl enzyme intermediate between the substrate and the essential Serine. Formation of this covalent intermediate proceeds through a negatively charged tetrahedral transition state intermediate and then the peptide bond is cleaved. During the second step or deacylation, the acyl-enzyme intermediate is hydrolyzed by a water molecule to release the peptide and to restore the Ser-hydroxyl of the enzyme. The deacylation which also involves the formation of a tetrahedral transition state intermediate, proceeds through the reverse reaction pathway of acylation. A water molecule is the attacking nucleophile instead of the Ser residue. The His residue provides a general base and accept the OH group of the reactive Ser.

B. Cysteine Endopeptidases

This family includes the plant proteases such as papain, actinidin or bromelain, several mammalian lysosomal cathepsins, the cytosolic calpains (calcium-activated) as well as several parasitic proteases (e.g., Trypanosoma, Schistosoma). Papain is the archetype and the best studied member of the family. Recent elucidation of the X-ray structure of the Interleukin-1-beta Converting Enzyme has revealed a novel type of fold for cysteine endopeptidases. Like the serine endopeptidases, catalysis proceeds through the formation of a covalent intermediate and involves a cysteine and a histidine residue. The essential Cys25 and His159 (papain numbering) play the same role as Ser195 and His57 respectively. The nucleophile is a thiolate ion rather than a hydroxyl group. The thiolate ion is stabilized through the formation of an ion pair with neighboring imidazolium group of His159. The attacking nucleophile is the thiolate-imidazolium ion pair in both steps and then a water molecule is not required.

C. Aspartic Endopeptidases

Most of aspartic endopeptidases belong to the pepsin family. The pepsin family includes digestive enzymes such as pepsin and chymosin as well as lysosomal cathepsins D and processing enzymes such as renin, and certain fungal proteases (penicillopepsin, rhizopuspepsin, endothiapepsin). A second family comprises viral endopeptidases such as the protease from the AIDS virus (HIV) also called retropepsin. Crystallographic studies have allowed to show that these enzymes are bilobed molecules with the active site located between two homologous lobes. Each lobe contributes one aspartate residue of the catalytically active diad of aspartates. These two aspartyl residues are in close geometric proximity in the active molecule and one aspartate is ionized whereas the second one is unionized at the optimum pH range of 2-3. Retropepsins, are monomeric, i.e., carry only one catalytic aspartate and then dimerization is required to form an active enzyme.

In contrast to serine and cysteine proteases, catalysis by aspartic endopeptidases do not involve a covalent intermediate though a tetrahedral intermediate exists. The nucleophilic attack is achieved by two simultaneous proton transfer: one from a water molecule to the diad of the two carboxyl groups and a second one from the diad to the carbonyl oxygen of the substrate with the concurrent CO—NH bond cleavage. This general acid-base catalysis, which may be called a “push-pull” mechanism leads to the formation of a non-covalent neutral tetrahedral intermediate.

D. Metallo Endopeptidases

The metallo endopeptidases may be one of the older classes of endopeptidases and are found in bacteria, fungi as well as in higher organisms. They differ widely in their sequences and their structures but the great majority of enzymes contain a zinc atom which is catalytically active. In some cases, zinc may be replaced by another metal such as cobalt or nickel without loss of the activity. Bacterial thermolysin has been well characterized and its crystallographic structure indicates that zinc is bound by two histidines and one glutamic acid. Many enzymes contain the sequence HEXXH, which provides two histidine ligands for the zinc whereas the third ligand is either a glutamic acid (thermolysin, neprilysin, alanyl aminopeptidase) or a histidine (astacin). Other families exhibit a distinct mode of binding of the Zn atom. The catalytic mechanism leads to the formation of a non covalent tetrahedral intermediate after the attack of a zinc-bound water molecule on the carbonyl group of the scissile bond. This intermediate is further decomposed by transfer of the glutamic acid proton to the leaving group.

E. Bacterial/Toxin Endopeptidases

Toxin endopeptidases, usually of bacterial origin, can have a devastating and sometime lethal impact on host organisms. Some of the better known bacterial endopeptidase toxin are listed below in Table 1.

TABLE 1
Bacterial Endopeptidases

| | | | Substrate | |
|---|---|---|---|---|
| | Mode of | Target | Homolog in | |
| Organism/Toxin | Action | (Cleavage Site) | Yeast | Disease |
| B. anthracis/lethal | Metalloprotease | MAPKK1/MAPKK2 | MEKK | Anthrax |
| factor | | (multiple) | Family | |
| C. botulinum/ | Zinc- | SNAP-25 | SEC9 | Botulism |
| neurotxin A | metalloprotease | (ANQ/RAT) | | |
| C. botulinum/ | Zinc- | VAMP/synaptobrevin | Snc1, Snc2 | Botulism |
| neurotxin B | metalloprotease | (ASQ/FET) | | |
| C. botulinum/ | Zinc- | Syntaxin | Sso1, Sso2 | Botulism |
| neurotxin C | metalloprotease | (TKK/AVK) | | |
| C. botulinum/ | Zinc- | VAMP/synaptobrevin | Snc1, Snc2 | Botulism |
| neurotxin D | metalloprotease | (DQK/LSE) | | |
| C. botulinum/ | Zinc- | SNAP-25 | SEC9 | Botulism |
| neurotxin E | metalloprotease | (IDR/IME) | | |
| C. botulinum/ | Zinc- | VAMP/synaptobrevin | Snc1, Snc2 | Botulism |
| neurotxin F | metalloprotease | (RDQ/KLS) | | |
| C. botulinum/ | Zinc- | VAMP/synaptobrevin | Snc1, Snc2 | Botulism |
| neurotxin G | metalloprotease | (TSA/AKL) | | |
| Yersinia virulence | Cysteine | Unknown | Unknown | Plague |
| factor YopJ | protease | | | |
| Yersinia virulence | Cysteine | Prenylated cysteine | Unknown | Plague |
| factor YopT | protease | | | |
| Salmonella virulence | unknown | Unknown | Unknown | Salmonellosis |
| factor AvrA | | | | |
| Clostridium | Zinc- | VAMP/synaptobrevin | Snc1, Snc2 | Tetanus |
| tetani/tetanus toxin | metalloprotease | (ASQ/FET) | | |

The C. botulinum neurotoxins (BoNTs, serotypes A-G) and the C. tetani tetanus neurotoxin (TeNT) are two examples of bacterial toxins that are endopeptidases. BoNTs are most commonly associated with infant and food-borne botulism and exist in nature as large complexes comprised of the neurotoxin and one or more associated proteins believed to provide protection and stability to the toxin molecule while in the gut. TeNT, which is synthesized from vegetative C. tetani in wounds, does not appear to form complexes with any other protein components.

The BoNTs and TeNT are either plasmid encoded (TeNT, BoNTs/A, G, and possibly B) or bacteriophage encoded (BoNTs/C, D, E, F), and the neurotoxins are synthesized as inactive polypeptides of 150 kDa (44). BoNTs and TeNT are released from lysed bacterial cells and then activated by the proteolytic cleavage of an exposed loop in the neurotoxin polypeptide. Each active neurotoxin molecule consists of a heavy (100 kDa) and light chain (50 kDa) linked by a single interchain disulphide bond. The heavy chains of both the BoNTs and TeNT contain two domains: a region necessary for toxin translocation located in the N-terminal half of the molecule, and a cell-binding domain located within the C-terminus of the heavy chain. The light chains of both the BoNTs and TeNT contain zinc-binding motifs required for the zinc-dependent protease activities of the molecules.

The cellular targets of the BoNTs and TeNT are a group of proteins required for docking and fusion of synaptic vesicles to presynaptic plasma membranes and therefore essential for the release of neurotransmitters. The BoNTs bind to receptors on the presynaptic membrane of motor neurons associated with the peripheral nervous system. Proteolysis of target proteins in these neurons inhibits the release of acetylcholine, thereby preventing muscle contraction. BoNTs/B, D, F, and G cleave the vesicle-associated membrane protein and synaptobrevin, BoNT/A and E target the synaptosomal-associated protein SNAP-25, and BoNT/C hydrolyzes syntaxin and SNAP-25. TeNT affects the central nervous system and does so by entering two types of neurons. TeNT initially binds to receptors on the presynaptic membrane of motor neurons but then migrates by retrograde vesicular transport to the spinal cord, where the neurotoxin can enter inhibitory interneurons. Cleavage of the vesicle-associated membrane protein and synaptobrevin in these neurons disrupts the release of glycine and gamma-amino-butyric acid, which, in turn, induces muscle contraction. The contrasting clinical manifestations of BoNT or TeNT intoxication (flaccid and spastic paralysis, respectively) are the direct result of the specific neurons affected and the type of neurotransmitters blocked.

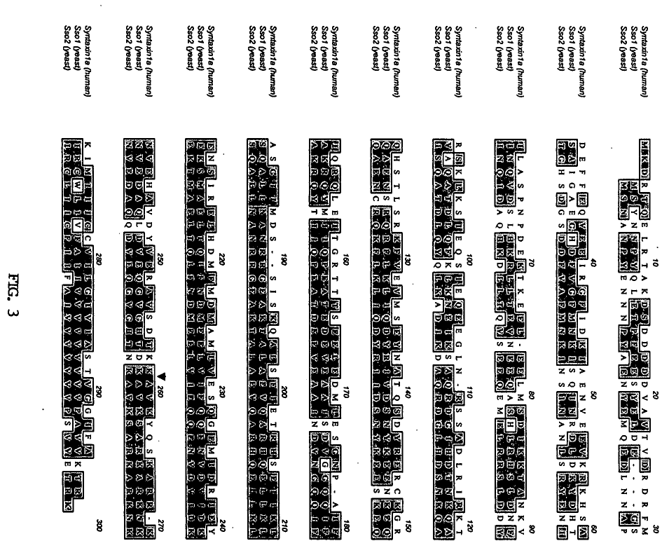

Of particular interest is BoNT/LC (serotype C), and specifically BoNTC/LC (as compared to other LC serotypes). First, BoNTC/LC poses a particularly significant bioterror threat because it has a long half-life inside human neuronal cells. Second, an in vitro assay for BoNTC/LC does not currently exist, probably because this LC protease appears to require membranes to function. In the neuronal cell environment, BoNTC/LC cleaves syntaxin, a membrane protein required for synaptic vesicle fusion to the presynaptic membrane. The yeast Saccharomyces cerevisiae has two functionally redundant homologs of syntaxin, Ssolp and Sso2. Sso1p and Sso2p perform the same required step in the fusion of secretory vesicles to the plasma membrane of yeast, indicating syntaxin exhibits functional similarities to Ssolp and Sso2p. As can be seen in FIG. 3, syntaxin exhibits strong sequence similarity to Ssolp and Sso2p, particularly at the syntaxin cleavage site (indicated by an arrow).

Other examples include the Yersinia virulence factors YopJ and YopT, as well as Salmonella AvrA.

F. Exopeptidases

Exopeptidases act only near the ends of polypeptide chains, and those acting at a free N-terminus liberate a single amino-acid residue (aminopeptidases), or a dipeptide or a tripeptide (dipeptidyl-peptidases and tripeptidyl-peptidases). The exopeptidases acting at a free C-terminus liberate a single residue (carboxypeptidases) or a dipeptide (peptidyl-dipeptidases). The carboxypeptidases are allocated to three groups on the basis of catalytic mechanism: the serine-type carboxypeptidases, the metallocarboxypeptidases and the cysteine-type carboxypeptidases. Other exopeptidases are specific for dipeptides (dipeptidases), or remove terminal residues that are substituted, cyclized or linked by isopeptide bonds (peptide linkages other than those of α-carboxyl to α-amino groups) (σ peptidases).

III. Candidate Endopeptidase Inhibitors

A. Known Inhibitors

Over 100 naturally-occurring protein protease inhibitors have been identified so far, thereby demonstrating the likelihood of finding additional endopeptidase inhibitors. They have been isolated in a variety of organisms from bacteria to animals and plants. They behave as tight-binding reversible or pseudo-irreversible inhibitors of proteases preventing substrate access to the active site through steric hindrance. Their size are also extremely variable from 50 residues (e.g., BPTI: Bovine Pancreatic Trypsin Inhibitor) to up to 400 residues (e.g., α-1PI: α-1 Endopeptidase Inhibitor). They are strictly class-specific except proteins of the alpha-macroglobulin family (e.g., α-2 macroglobulin) which bind and inhibit most proteases through a molecular trap mechanism.

Serine protease inhibitors have been the most studied protein inhibitors up to know and recently a considerable advance has been made in the study of the natural inhibitors of cysteine proteases (cystatins). Some other endopeptidase inhibitors include Amastatin, E-64, Antipain, Elastatinal, APMSF, Leupeptin, Bestatin, Pepstatin, Benzamidine, 1,10-Phenanthroline, Chymostatin, Phosphoramidon, 3,4-dichloroisocoumarin, TLCK, DFP and TPCK.

B. Natural and Synthetic Inhibitors

As used herein, the term “candidate inhibitor” refers to any molecule that may potentially reduce endopeptidase cleavage. The candidate may be a protein or fragment thereof, a small molecule, or even a nucleic acid molecule. It may prove to be the case that the most useful pharmacological compounds will be compounds that are structurally related to compounds which interact naturally with endopeptidases. Creating and examining the action of such molecules is known as “rational drug design,” and include making predictions relating to the structure of the target molecules and the candidate substance.

The goal of rational drug design is to produce structural analogs of biologically active polypeptides or target compounds. By creating such analogs, it is possible to fashion drugs which are more active or stable than the natural molecules, which have different susceptibility to alteration or which may affect the function of various other molecules. In one approach, one would generate a three-dimensional structure for a molecule like an endopeptidase, and then design a molecule for its ability to interact with these polypeptides. Alternatively, one could design a partially functional fragment of these polypeptides (binding, but no activity), thereby creating a competitive inhibitor. This could be accomplished by x-ray crystallography, computer modeling or by a combination of both approaches.

It also is possible to use antibodies to ascertain the structure of a target compound. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds or may be found as active combinations of known compounds which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds.

Yet further, the candidate substance may be a known antibiotic. The term “antibiotics” as used herein is defined as a substance that inhibits the growth of microorganisms without equivalent damage to the host. Yet further, it is within the scope of the present invention to synthesis or produce analogs of known antibiotics. These analogs may have been altered, for example site-directed mutagenesis, to exhibit increased antimicrobial activity.

It will, of course, be understood that all the screening methods of the present invention are useful in themselves notwithstanding the fact that effective candidates may not be found. The invention provides methods for screening for such candidates, not solely methods of finding them.

IV. Yeast

Yeast are unicellular fungi whose mechanisms of cell-cycle control are remarkably similar to that of humans. The precise classification is a field that uses the characteristics of the cell, ascospore and colony. Physiological characteristics are also used to identify species. One of the more well known characteristics is the ability to ferment sugars for the production of ethanol. Budding yeasts are true fungi of the phylum Ascomycetes, class Hemiascomycetes. The true yeasts are separated into one main order Saccharomycetales. Yeasts are characterized by a wide dispersion of natural habitats, and are common on plant leaves and flowers, soil and salt water. Yeasts are also found on the skin surfaces and in the intestinal tracts of warm-blooded animals, where they may live symbiotically or as parasites.

Yeasts multiply as single cells that divide by budding (e.g., Saccharomyces) or direct division (fission, e.g., Schizosaccharomyces), or they may grow as simple irregular filaments (mycelium). In sexual reproduction most yeasts form asci, which contain up to eight haploid ascospores. These ascospores may fuse with adjoining nuclei and multiply through vegetative division or, as with certain yeasts, fuse with other ascospores.

The awesome power of yeast genetics is partially due to the ability to quickly map a phenotype producing gene to a region of the S. cerevisiae genome. For the past two decades, S. cerevisiae has been the model system for much of molecular genetic research because the basic cellular mechanics of replication, recombination, cell division and metabolism are generally conserved between yeast and larger eukaryotes, including mammals. It is also a straightforward matter to engineer yeast cells to express a variety of heterologous constructs, and to do so in a controlled fashion.

A. Yeast Cultures

Some yeast varieties reproduce almost as rapidly as bacteria and have a genome size less than 1% that of a mammal. They are amenable to rapid molecular genetic manipulation, whereby genes can be deleted, replaced, or altered. They also have the unusual ability to proliferate in a haploid state, in which only a single copy of each gene is present in the cell. This makes it easy to isolate and study mutations that inactivate a gene as one avoids the complication of having a second copy of the gene in the cell.

The process of culturing yeast strains involves isolation of a single yeast cell, maintenance of yeast cultures, and the propagation of the yeast until an amount sufficient for pitching is obtained. Pure yeast cultures are obtained from a number of sources such as commercial distributors or culture collections. Various procedures are used to collect pure cultures, including culturing from a single colony, a single cell, or a mixture of isolated cells and colonies.

The objective of propagation is to produce large quantities of yeast with known characteristics in as short a time as possible. One method is a batch system of propagation, starting with a few milliliters of stock culture and scaling up until a desired quantity of yeast has been realized. Scale-up introduces actively growing cells to a fresh supply of nutrients in order to produce a crop of yeast in the optimum physiological state.

Yeast cells that may be used in accordance with the present invention include, but are not limited to, Saccharomyses species (e.g., S. cerevisiae; S. carlsbergensis), Schizosaccharomyces species (e.g., S. pombi), Pichia species (e.g., P. pastoris), Hansenula species (e.g., H. polymorpha), Kluyveromyces species (e.g., K. lactis), Yarrowia species (e.g., Y. lipolytica). However, virtually any yeast cell genus can be engineered for sensitivity to bacterial toxins as described herein.

B. Yeast Viability and Growth

The adoption of means to enhance vector stability increases the yield of the expression product from a culture. Many vectors adapted for cloning in yeast include genetic markers to insure growth of transformed yeast cells under selection pressure. Host cell cultures containing such vectors may contain large numbers of untransformed segregants when grown under nonselective conditions, especially when grown to high cell densities. Therefore, it is advantageous to employ expression vectors which do not require growth under selection conditions, in order to permit growth to high densities and to minimize the proportion of untransformed segregants.

Vectors which contain a substantial portion of the naturally-occurring two circle plasmid are able to replicate stably with minimal segregation of untransformed cells, even at high cell densities, when transformed into host strains previously lacking two micron circles. Such host strains are termed “circle zero” strains. Additionally, the rate of cell growth at low cell densities may be enhanced by incorporating regulatory control over the promoter such that the expression of the S-protein coding region is minimized in dilute cultures such as early to middle log phase, then turned on for maximum expression at high cell densities. Such a control strategy increases the efficiency of cell growth in the fermentation process and further reduces the frequency of segregation of untransformed cells.

Briefly, yeast may be transfected with an expression vector expressing an essential polypeptide that has been engineered to include an endopeptidase cleavage site. Rates of growth in liquid medium of transformed yeast may be measured in the presence of galactose, which induces expression. Viability is a measure of yeast's ability to ferment. Yeast viability is determined by the standard-culture method, flow cytometry by selective staining, or by more advanced methods such as the Slide Viability Method, flocculation tests, and fermentation tests.

The standard slide-culture method of determining viability of yeasts has three steps: perform a hemacytometer count on a suspension of cells, plate a measured quantity on a wort gelatin medium, and then incubate and count the resultant colonies. However, this method may be inaccurate due to cell clumping and the death of cells during preparation.

Methylene blue remains an industry standard for viability assessment. It has also been suggested that methylene violet might provide a more accurate and reproducible assessment of viability than does methylene blue because of impurities in the latter. Other stains that may be used include fluorophore dyes, such as oxonol (DiBAC), 1-anilino-8-naphtalene-sulfonic acid (MgANS), berberine, Sytox Orange, propidium iodide, FUN1, and other conventional brightfield dyes. For the most part, fluorophore staining has been perceived to be less subjective to the operator compared with brightfield dye staining because of the lack of intermediate color variations.

C. Yeast Promoters

Useful yeast promoters for the conditional expression of toxic peptidases include those directing expression of metallothionein, 3-phosphoglycerate kinase or other glycolytic enzymes such as enolase or glyceraldehyde-3-phosphate dehydrogenase, enzymes responsible for maltose and galactose utilization, and others. Vectors and promoters suitable for use in yeast expression are further described in EP 73,675A, herein incorporated by reference in its entirety. Other examples of strong yeast promoters are the alcohol dehydrogenase, lactase and triosephosphate isomerase promoters

For expression of yeast genes in yeast, to determine the effects of mutations, it is generally best to use the gene's promoter in a CEN plasmid so expression is similar to the wild-type gene. However, there are a variety of promoters to choose from for various purposes. One such promoter is the Gal 1,10 promoter, which is inducible by galactose. It is frequently valuable to be able to turn expression of the gene on and off so one can follow the time dependent effects of expression.

The Gal 1 gene and Gal 10 gene are adjacent and transcribed in opposite directions from the same promoter region. The regulatory region containing the UAS sequences can be cut out on a DdeI Sau3A fragment and placed upstream of any other gene to confer galactose inducible expression and glucose repression. The PGK, GPD and ADH1 promoters are high expression constitutive promoters (PGK=phosphoglycerate kinase, GPD=glyceraldehyde 3 phosphate dehydrogenase, ADH1=alcohol dehydrogenase). The ADH2 promoter is glucose repressible and it is strongly transcribed on non-fermentable carbon sources (similar to GAL 1 or 10) except not inducible by galactose. The CUPI promoter is the metalothionein gene promoter. It is activated by copper or silver ions added to the medium. The CUP 1 gene is one of a few yeast genes that is present in yeast in more than one copy. Depending on the strain, there can be up to eight copies of this gene. The PHO5 promoter is a secreted gene coding for an acid phosphatase. It is induced by low or no phosphate in the medium. The phosphatase is secreted in the chance it will be able to free up some phosphate from the surroundings. When phosphate is present, no PHO5 message can be found. When it is absent, it is turned on strongly.

D. Non-Yeast Inducible Promoters

The identity of tissue-specific promoters or elements is well known to those of skill in the art. Nonlimiting examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), DIA dopamine receptor gene (Lee, et al., 1997), insulin-like growth factor II (Wu et al., 1997), and human platelet endothelial cell adhesion molecule-1 (Almendro et al., 1996) and the Tet-On™ and Tet-Off™ Systems from Clontech. Additional inducible promoters are discussed Table 2, below.

TABLE 2
Inducible Elements

| Element | Inducer | References |
|---|---|---|
| MT II | Phorbol Ester (TFA) | Palmiter et al., 1982; Haslinger |
| | Heavy metals | et al., 1985; Searle et al., 1985; |
| | | Stuart et al., 1985; Imagawa |
| | | et al., 1987, Karin et al., 1987; |
| | | Angel et al., 1987b; McNeall |
| | | et al., 1989 |
| MMTV (mouse mammary | Glucocorticoids | Huang et al., 1981; Lee et al., |
| tumor virus) | | 1981; Majors et al., 1983; |
| | | Chandler et al., 1983; Lee et al., |
| | | 1984; Ponta et al., 1985; Sakai |
| | | et al., 1988 |
| β-Interferon | Poly(rI)x | Tavernier et al., 1983 |
| | Poly(rc) | |
| Adenovirus 5 E2 | E1A | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene H-2κb | Interferon | Blanar et al., 1989 |
| HSP70 | E1A, SV40 Large T | Taylor et al., 1989, 1990a, 1990b |
| | Antigen | |
| Proliferin | Phorbol Ester-TPA | Mordacq et al., 1989 |
| Tumor Necrosis Factor α | PMA | Hensel et al., 1989 |
| Thyroid Stimulating | Thyroid Hormone | Chatterjee et al., 1989 |
| Hormone α Gene | | |

E. Yeast Transformation Protocols

A variety of approaches are available for transforming yeast cells and include electroporation, lithium acetate and protoplasting. In certain embodiments of the present invention, a nucleic acid is introduced into an organelle, a cell, a tissue or an organism via electroporation. Electroporation involves the exposure of a suspension of cells and DNA to a high-voltage electric discharge. In some variants of this method, certain cell wall-degrading enzymes, such as pectin-degrading enzymes, are employed to render the target recipient cells more susceptible to transformation by electroporation than untreated cells (U.S. Pat. No. 5,384,253, incorporated herein by reference). Alternatively, recipient cells can be made more susceptible to transformation by mechanical wounding.

Protoplast fusion has been used to overcome sexual barriers that prevent genetically unrelated strains from mating (Svoboda, 1976), thus facilitating the total or partial exchange of genetic components (Provost et al., 1978; Wilson et al., 1982; Perez et al., 1984; Spencer et al., 1985; Pina et al., 1986; Skala et al., 1988; Janderova et al., 1990; Gupthar, 1992; Molnar and Sipiczki, 1993). The process relies on cell wall digestion followed by fusion with, e.g., polyethylene glycol (Kao and Michayluk, 1974) and the protoplast adhesion promoter, Ca²⁺ have been exploited in yeast fusion experiments (van Solingen and van der Plaat, 1977; Svoboda, 1978; Wilson et al., 1982; Pina et al., 1986). Other workers report “an enhancement of the protoplast fusion rate” using electro-fusion techniques instead of polyethylene glycol (Weber et al., 1981; Halfmann et al., 1982). The action of polyethylene glycol is not specific. It catalyses the aggregation of protoplasts between the same or different species.

The fusion process may be summarized as follows: (i) random aggregation of protoplasts into clumps of various sizes (Anne and Peberdy, 1975; Sarachek and Rhoads, 1981); (ii) conversion of the aggregates into syncytia (“chimaeric protoplast fusion product”) by dissolution of membranes and merging of cytoplasmic contents (Ahkong et al., 1975a; Gumpert, 1980; Svoboda, 1981; Sarachek and Rhoads, 1981; Klinner and Bottcher, 1984); (iii) membrane re-organisation (Ahkong et al., 1975a; Gumpert, 1980) and fusion of nuclei within heterokaryons (Sarachek and Rhoads, 1981; Klinner and Bottcher, 1984).

Another approach uses electroporation. Cells are first grown to a density of about 1×10⁷/ml (OD595 ca. 0.5) in minimal medium (transformation frequency is not harmed by growth until early stationary phase (OD595=1.5)). Cells are harvested by spinning at 3000 rpm for 5 minutes at 20° C., followed by washing once in ice-cold water and harvesting; a second time in ice-cold 1M sorbitol. It has been reported (Suga and Hatakeyama, 2001), that 15 min incubation of these cells in the presence of DTT at 25 mM increases electrocompetence. The final resuspension is in ice-cold 1M sorbitol at a density of 1-5×10⁹/ml. Forty ul of the cell suspension are added to chilled eppendorfs containing the DNA for transformation (100 ng) and incubated on ice for 5 minutes.

The electroporator may be set as follows: (a) 1.5 kV, 200 ohms, 25 uF (Biorad); (b) 1.5 kV, 132 ohms, 40 uF (Jensen/Flowgen). Cells and DNA are transferred to a pre-chilled cuvette and pulsed; 0.9 ml of ice-cold 1M sorbitol is then immediately added to the cuvette; the cell suspension is then returned to the eppendorf and placed on ice while other electroporations are carried out. Cells are plated as soon as possible onto minimal selective medium. Transformants should appear in 4-6 days at 32° C. The following lithium acetate protocol is derived from Okazaki et al. (1990), High-frequency transformation method and library transducing vectors for cloning mammalian cDNAs by trans-complementation of Schizosaccharomyces pombe. Cells are grown in a 150 ml culture in minimal medium to a density of 0.5-1×10⁷ cells/ml (OD595=0.2-0.5). Media with low glucose, or MB media (see Okazaki et al.), in which the cells are less happy, may increase transformation efficiency. Cells are harvested at 3000 rpm for 5 minutes at room temperature, then washed in 40 ml of sterile water and spun down as before. The cells are resuspend at 1×10⁹ cells/ml in 0.1 M lithium acatate (adjusted to pH 4.9 with acetic acid) and dispensed in 100 ul aliquots into eppendorf tubes. Incubation is at 30° C. (25° C. for ts mutants) for 60-120 min. Cells will sediment at this stage. One ug of plasmid DNA in 15 ul TE (pH 7.5) is added to each tube and mix by gentle vortexing, completely resuspending cells sedimented during the incubation. The tubes should not be allowed to cool down at this stage. 290 μl of 50% (w/v) PEG 4000 prewarmed at 30° C. (25° C. for ts mutants) is added. Next, mix by gentle vortexing and incubate at 30° C. (25° C. for ts mutants) for 60 minutes. The tubes are heat shocked at 43° C. for 15 minutes, followed by cooling to room temperature for 10 minutes. The tubes are then centrifuged at 5000 rpm for 2 minutes in an eppendorf centrifuge. The supernatant is carefully removed by aspiration. Cells are resuspend in 1 ml of ½ YE broth by pipetting up and down with a pipetman P1000, transferred to a 50 ml flask and diluted with 9 ml of ½ YE. The cells are incubated with shaking at 32° C. (25° C. for ts mutants) for 60 minutes or longer. Aliquots of less than 0.3 ml are plated onto minimal plates. If necessary, cells are centrifuged at this stage and resuspended in 1 ml of media to spread more cells on a plate.

F. Yeast Essential Genes

An “essential” yeast gene is defined as one that is imperative for the vegetative life cycle of a yeast cell grown on rich YPD media at 30° C. Over 800 essential yeast genes have been identified thus far. At present 16-18% of all yeast genes are essential for growth by the following definition. This number is probably an underestimation due to the huge number of gene families and the fact that many non-essential genes might become essential once functionally redundant genes have been deleted. This phenotype is termed synthetic lethality. The following table lists yeast essential genes which may be modified in accordance with the present invention.

TABLE 3
Yeast Essential Genes

| ORF | Name | Description |
|---|---|---|
| YOL038w | PRE6 | 20S proteasome subunit (alpha4) |
| YJL001w | PRE3 | 20S proteasome subunit (beta1) |
| YOR157c | PUP1 | 20S proteasome subunit (beta2) |
| YER094c | PUP3 | 20S proteasome subunit (beta3) |
| YPR103w | PRE2 | 20S proteasome subunit (beta5) |
| YOR362c | PRE10 | 20S proteasome subunit C1 (alpha7) |
| YER012w | PRE1 | 20S proteasome subunit C11 (beta4) |
| YML092c | PRE8 | 20S proteasome subunit Y7 (alpha2) |
| YGL011c | SCL1 | 20S proteasome subunit YC7ALPHA/Y8 (alpha1) |
| YGR253c | PUP2 | 20S proteasome subunit (alpha5) |
| YMR314w | PRE5 | 20S proteasome subunit (alpha6) |
| YBL041w | PRE7 | 20S proteasome subunit (beta6) |
| YFR050c | PRE4 | 20S proteasome subunit (beta7) |
| YDL007w | RPT2 | 26S proteasome regulatory subunit |
| YDR394w | RPT3 | 26S proteasome regulatory subunit |
| YER021w | RPN3 | 26S proteasome regulatory subunit |
| YFR004w | RPN11 | 26S proteasome regulatory subunit |
| YFR052w | RPN12 | 26S proteasome regulatory subunit |
| YGL048c | RPT6 | 26S proteasome regulatory subunit |
| YHR027c | RPN1 | 26S proteasome regulatory subunit |
| YIL075c | RPN2 | 26S proteasome regulatory subunit |
| YKL145w | RPT1 | 26S proteasome regulatory subunit |
| YOR259c | RPT4 | 26S proteasome regulatory subunit |
| YGR195w | SKI6 | 3′->5′ exoribonuclease required for 3′ end formation of 5.8S rRNA |
| YHR069c | RRP4 | 3′->5′ exoribonuclease required for 3′ end formation of 5.8S rRNA |
| YLR100w | ERG27 | 3-keto sterol reductase |
| YBR265w | TSC10 | 3-ketosphinganine reductase |
| YOL040c | RPS15 | 40S small subunit ribosomal protein |
| YOR048c | RAT1 | 5′-3′ exoribonuclease |
| YHR183w | GND1 | 6-phosphogluconate dehydrogenase |
| YLR075w | RPL10 | 60S large subunit ribosomal protein |
| YLR029c | RPL15A | 60s large subunit ribosomal protein L15.e.c12 |
| YOR063w | RPL3 | 60S large subunit ribosomal protein L3.e |
| YGL030w | RPL30 | 60S large subunit ribosomal protein L30.e |
| YBL092w | RPL32 | 60S large subunit ribosomal protein L32.e |
| YPL131w | RPL5 | 60S large subunit ribosomal protein L5.e |
| YJL085w | EXO70 | 70 kDa exocyst component protein |
| YPL028w | ERG10 | acetyl-CoA C-acetyltransferase, cytosolic |
| YNR016c | ACC1 | acetyl-CoA carboxylase |
| YLR340w | RPP0 | acidic ribosomal protein L10.e |
| YFL039c | ACT1 | actin |
| YBR211c | AME1 | actin related protein |
| YJR065c | ARP3 | actin related protein |
| YIR006c | PAN1 | actin-cytoskeleton assembly protein |
| YDL029w | ARP2 | actin-like protein |
| YJL081c | ARP4 | actin-related protein |
| YMR033w | ARP9 | Actin-related protein |
| YOL052c | SPE2 | adenosylmethionine decarboxylase precursor |
| YJL005w | CYR1 | adenylate cyclase |
| YOR335c | ALA1 | alanyl-tRNA synthetase, cytosolic |
| YBR126c | TPS1 | alpha,alpha-trehalose-phosphate synthase, 56 KD subunit |
| YML085c | TUB1 | alpha-1 tubulin |
| YDR341c | | arginyl-tRNA synthetase, cytosolic |
| YBR234c | ARC40 | ARP2/3 protein complex subunit, 40 kilodalton |
| YKL112w | ABF1 | ARS-binding factor |
| YHR019c | DED81 | asparaginyl-tRNA-synthetase |
| YLL018c | DPS1 | aspartyl-tRNA synthetase, cytosolic |
| YMR309c | NIP1 | associated with 40s ribosomal subunit |
| YLR298c | YHC1 | associated with the U1 snRNP complex |
| YGR013w | SNU71 | associated with U1 snRNP, no counterpart in mammalian U1 |
| | | snRNP |
| YMR301c | ATM1 | ATP-binding cassette transporter protein, mitochondrial |
| YBR142w | MAK5 | ATP-dependent RNA helicase |
| YGL171w | ROK1 | ATP-dependent RNA helicase |
| YOR204w | DED1 | ATP-dependent RNA helicase |
| YFL002c | SPB4 | ATP-dependent RNA helicase of DEAH box family |
| YIL048w | NEO1 | ATPase whose overproduction confers neomycin resistance |
| YKL004w | AUR1 | aureobasidin-resistance protein |
| YMR308c | PSE1 | beta karyopherin |
| YBR110w | ALG1 | beta-mannosyltransferase |
| YFL037w | TUB2 | beta-tubulin |
| YDL141w | BPL1 | biotin holocarboxylase synthetase |
| YLR116w | MSL5 | branch point bridging protein |
| YNL280c | ERG24 | C-14 sterol reductase |
| YGL001c | ERG26 | C-3 sterol dehydrogenase (C-4 decarboxylase) |
| YGR060w | ERG25 | C-4 sterol methyl oxidase |
| YBR109c | CMD1 | calmodulin |
| YJL033w | HCA4 | can suppress the U14 snoRNA rRNA processing function |
| YPL204w | HRR25 | casein kinase I, ser/thr/tyr protein kinase |
| YFL029c | CAK1 | cdk-activating protein kinase |
| YPR113w | PIS1 | CDP diacylglycerol-inositol 3-phosphatidyltransferase |
| YER026c | CHO1 | CDP-diacylglycerol serine O-phosphatidyltransferase |
| YBR136w | MEC1 | cell cycle checkpoint protein |
| YDR499w | LCD1 | cell cycle checkpoint protein |
| YDR113c | PDS1 | cell cycle regulator |
| YOR373w | NUD1 | cell cycle regulatory protein |
| YBR202w | CDC47 | cell division control protein |
| YDL220c | CDC13 | cell division control protein |
| YDR168w | CDC37 | cell division control protein |
| YDR182w | CDC1 | cell division control protein |
| YFL009w | CDC4 | cell division control protein |
| YGL116w | CDC20 | cell division control protein |
| YJL194w | CDC6 | cell division control protein |
| YLR274w | CDC46 | cell division control protein |
| YLR314c | CDC3 | cell division control protein |
| YNL188w | KAR1 | cell division control protein |
| YPL255w | BBP1 | cell division control protein |
| YOL123w | HRP1 | CF Ib (RNA3′ Cleavage factor Ib) |
| YJL142w | CCT2 | chaperonin of the TCP1 ring complex, cytosolic |
| YIL014w | CCT3 | chaperonin of the TCP1 ring complex, cytosolic |
| YOR020c | HSP10 | chaperonin, mitochondrial |
| YFL008w | SMC1 | chromosome segregation protein |
| YFR031c | SMC2 | chromosome segregation protein |
| YLR115w | CFT2 | cleavage and polyadenylation specificity factor, part of CF II |
| YOR250c | CLP1 | cleavage/polyadenylation factor IA subunit |
| YDL145c | COP1 | coatomer complex alpha chain of secretory pathway vesicles |
| YDR238c | SEC26 | coatomer complex beta chain of secretory pathway vesicles |
| YGL137w | SEC27 | coatomer complex beta′ chain (beta′-cop) of secretory pathway |
| | | vesicles |
| YFR051c | RET2 | coatomer complex delta chain |
| YNL287w | SEC21 | coatomer complex gamma chain (gamma-COP) of secretory |
| | | pathway vesicles |
| YLL050c | COF1 | cofilin, actin binding and severing protein |
| YGL029w | CGR1 | Coiled-coil protein, may play a role in ribosome biogenesis |
| YMR288w | HSH155 | component of a multiprotein splicing factor |
| YDL143w | CCT4 | component of chaperonin-containing T-complex |
| YDR212w | TCP1 | component of chaperonin-containing T-complex |
| YDR188w | CCT6 | component of chaperonin-containing T-complex (zeta subunit) |
| YIL109c | SEC24 | component of COPII coat of ER-Golgi vesicles |
| YDR170c | SEC7 | component of non-clathrin vesicle coat |
| YDR228c | PCF11 | component of pre-mRNA 3′-end processing factor CF I |
| YGL044c | RNA15 | component of pre-mRNA 3′-end processing factor CF I |
| YMR061w | RNA14 | component of pre-mRNA 3′-end processing factor CF I |
| YJR093c | FIP1 | component of pre-mRNA polyadenylation factor PF I |
| YLR277c | YSH1 | component of pre-mRNA polyadenylation factor PF I |
| YPR056w | TFB4 | component of RNA polymerase transcription initiation TFIIH |
| | | factor |
| YPR034w | ARP7 | component of SWI-SNF global transcription activator complex and |
| | | RSC chromatin remodeling complex |
| YCR042c | TSM1 | component of TFIID complex |
| YHR099w | TRA1 | component of the Ada-Spt transcriptional regulatory complex |
| YLR127c | APC2 | component of the anaphase promoting complex |
| YOR249c | APC5 | component of the anaphase-promoting complex |
| YDL195w | SEC31 | component of the COPII coat of ER-golgi vesicles |
| YKL049c | CSE4 | component of the core centromere |
| YPL011c | TAF47 | component of the TBP-associated protein complex |
| YMR028w | TAP42 | component of the Tor signaling pathway |
| YHR148w | IMP3 | component of the U3 small nucleolar ribonucleoprotein |
| YJR002w | MPP10 | component of the U3 small nucleolar ribonucleoprotein |
| YNL075w | IMP4 | component of the U3 small nucleolar ribonucleoprotein YDL132w |
| CDC53 | | controls G1/S transition |
| YNL232w | CSL4 | core component of the 3′-5′ exosome |
| YOR206w | NOC2 | crucial for intranuclear movement of ribosomal precursor particles |
| YBR135w | CKS1 | cyclin-dependent kinases regulatory subunit |
| YBR160w | CDC28 | cyclin-dependent protein kinase |
| YDL108w | KIN28 | cyclin-dependent ser/thr protein kinase |
| YNL247w | | cysteinyl-tRNA synthetase |
| YHR007c | ERG11 | cytochrome P450 lanosterol 14a-demethylase |
| YER023w | PRO3 | delta 1-pyrroline-5-carboxylate reductase |
| YHR068w | DYS1 | deoxyhypusine synthase |
| YAL034w-a | MTW1 | determining metaphase spindle length |
| YMR113w | FOL3 | dihydrofolate synthetase |
| YNL256w | FOL1 | Dihydroneopterin aldolase, dihydro-6-hydroxymethylpterin |
| | | pyrophosphokinase, dihydropteroate synthetase |
| YJR016c | ILV3 | dihydroxy-acid dehydratase |
| YIL144w | TID3 | DMC1P interacting protein |
| YHR164c | DNA2 | DNA helicase |
| YIL143c | SSL2 | DNA helicase |
| YER171w | RAD3 | DNA helicase/ATPase |
| YJL173c | RFA3 | DNA replication factor A, 13 KD subunit |
| YNL312w | RFA2 | DNA replication factor A, 36 kDa subunit |
| YAR007c | RFA1 | DNA replication factor A, 69 KD subunit |
| YOLO094c | RFC4 | DNA replication factor C, 37 kDa subunit |
| YBR087w | RFC5 | DNA replication factor C, 40 KD subunit |
| YNL290w | RFC3 | DNA replication factor C, 40 kDa subunit |
| YJR068w | RFC2 | DNA replication factor C, 41 KD subunit |
| YOR217w | RFC1 | DNA replication factor C, 95 KD subunit |
| YNL088w | TOP2 | DNA topoisomerase II (ATP-hydrolysing) |
| YNL216w | RAP1 | DNA-binding protein with repressor and activator activity |
| YKL144c | RPC25 | DNA-direcred RNA polymerase III, 25 KD subunit |
| YKL045w | PRI2 | DNA-directed DNA polymerase alpha, 58 KD subunit (DNA |
| | | primase) |
| YIR008c | PRI1 | DNA-directed DNA polymerase alpha 48 kDa subunit (DNA |
| | | primase) |
| YNL102w | POL1 | DNA-directed DNA polymerase alpha, 180 KD subunit |
| YBL035c | POL12 | DNA-directed DNA polymerase alpha, 70 KD subunit |
| YJR006w | HYS2 | DNA-directed DNA polymerase delta, 55 KD subunit |
| YDL102w | CDC2 | DNA-directed DNA polymerase delta, catalytic 125 KD subunit |
| YNL262w | POL2 | DNA-directed DNA polymerase epsilon, catalytic subunit A |
| YPR175w | DPB2 | DNA-directed DNA polymerase epsilon, subunit B |
| YOR210w | RPB10 | DNA-directed polymerase I, II, III 8.3 subunit |
| YPR010c | RPA135 | DNA-directed RNA polymerase I, 135 KD subunit |
| YOR341w | RPA190 | DNA-directed RNA polymerase I, 190 KD alpha subunit |
| YOR340c | RPA43 | DNA-directed RNA polymerase I, 36 KD subunit |
| YOR224c | RPB8 | DNA-directed RNA polymerase I, II, III 16 KD subunit |
| YPR187w | RPO26 | DNA-directed RNA polymerase I, II, III 18 KD subunit |
| YBR154c | RPB5 | DNA-directed RNA polymerase I, II, III 25 KD subunit |
| YPR110c | RPC40 | DNA-directed RNA polymerase I, III 40 KD subunit |
| YNL113w | RPC19 | DNA-directed RNA polymerase I, III 16 KD subunit |
| YDR308c | SRBY | DNA-directed RNA polymerase II holoenzyme and kornberg's |
| | | mediator (SRB) subcomplex subunit |
| YER022w | SRB4 | DNA-directed RNA polymerase II holoenzyme and Kornberg's |
| | | mediator (SRB) subcomplex subunit |
| YLR071c | RGR1 | DNA-directed RNA polymerase II holoenzyme subunit |
| YOL005c | RPB11 | DNA-directed RNA polymerase II subunit, 13.6 kD |
| YBR253w | SRB6 | DNA-directed RNA polymerase II suppressor protein |
| YOR151c | RPB2 | DNA-directed RNA polymerase II, 140 kDa chain |
| YDR404c | RPB7 | DNA-directed RNA polymerase II, 19 KD subunit |
| YDL140c | RPO21 | DNA-directed RNA polymerase II, 215 KD subunit |
| YOR207c | RET1 | DNA-directed RNA polymerase III, 130 KD subunit |
| YOR116c | RPO31 | DNA-directed RNA polymerase III, 160 KD subunit |
| YNL151c | RPC31 | DNA-directed RNA polymerase III, 31 KD subunit |
| YNR003c | RPC34 | DNA-directed RNA polymerase III, 34 KD subunit |
| YDL150w | RPC53 | DNA-directed RNA polymerase III, 47 KD subunit |
| YPR190c | RPC82 | DNA-directed RNA polymerase III, 82 KD subunit |
| YHR143w-a | RPC10 | DNA-directed RNA polymerases I, II, III 7.7 KD subunit |
| YIL021w | RPB3 | DNA-directed RNA-polymerase II, 45 kDa |
| YMR013c | SEC59 | dolichol kinase |
| YPR183w | DPM1 | dolichyl-phosphate beta-D-mannosyltransferase |
| YLR129w | DIP2 | DOM34P-interacting protein |
| YMR239c | RNT1 | double-stranded ribonuclease |
| YOL066c | RIB2 | DRAP deaminase |
| YJR057w | CDC8 | dTMP kinase |
| YFR028c | CDC14 | dual specificity phosphatase |
| YBR252w | DUT1 | dUTP pyrophosphatase precursor |
| YDR390c | UBA2 | E1-like (ubiquitin-activating) enzyme |
| YKL210w | UBA1 | E1-like (ubiquitin-activating) enzyme |
| YDL064w | UBC9 | E2 ubiquitin-conjugating enzyme |
| YDR054c | CDC34 | E2 ubiquitin-conjugating enzyme |
| YBR247c | ENP1 | effects N-glycosylation |
| YBL040c | ERD2 | ER lumen protein-retaining receptor |
| YDR086c | SSS1 | ER protein-translocase complex subunit |
| YLR378c | SEC61 | ER protein-translocation complex subunit |
| YOR254c | SEC63 | ER protein-translocation complex subunit |
| YPL094c | SEC62 | ER protein-translocation complex subunit |
| YFL017c | GNA1 | essential acetyltransferase |
| YDR331w | GPI8 | essential for GPI anchor attachment |
| YGR113w | DAM1 | essential mitotic spindle pole protein |
| YGL061c | DUO1 | essential mitotic spindle protein |
| YIL026c | IRR1 | essential protein |
| YDR473c | PRP3 | essential splicing factor |
| YEL020w-a | TIM9 | essential subunit of the TIM22-complex for mitochondrial protein |
| | | import |
| YOR319w | HSH49 | essential yeast splicing factor |
| YDR172w | SUP35 | eukaryotic peptide chain release factor GTP-binding subunit |
| YBR102c | EXO84 | exocyst protein essential for secretion |
| YPL169c | MEX67 | factor for nuclear mRNA export |
| YHR190w | ERG9 | farnesyl-diphosphate farnesyltransferase |
| YJL167w | ERG20 | farnesyl-pyrophosphate synthetase |
| YPL231w | FAS2 | fatty-acyl-CoA synthase, alpha chain |
| YKL182w | FAS1 | fatty-acyl-CoA synthase, beta chain |
| YDL014w | NOP1 | fibrillarin |
| YDL045c | FAD1 | flavin adenine dinucleotide (FAD) synthetase |
| YMR203w | TOM40 | forms the hydrophilic channel of the mitochondrial import pore for |
| | | preproteins |
| YPR180w | AOS1 | forms together with |
| UBA2P | | a heterodimeric activating enzyme for SMT3P |
| YKL060c | FBA1 | fructose-bisphosphate aldolase |
| YDR397c | NCB2 | functional homolog of human NC2beta/Dr1 |
| YLR212c | TUB4 | gamma tubulin |
| YER136w | GDI1 | GDP dissociation inhibitor |
| YGL225w | GOG5 | GDP-mannose transporter into the lumen of the Golgi |
| YGL097w | SRM1 | GDP/GTP exchange factor for GSP1P/GSP2P |
| YLR310c | CDC25 | GDP/GTP exchange factor for RAS1P and RAS2P |
| YGL207w | SPT16 | general chromatin factor |
| YJL031c | BET4 | geranylgeranyl transferase, alpha chain |
| YGL155w | CDC43 | geranylgeranyltransferase beta subunit |
| YOR370c | MRS6 | geranylgeranyltransferase regulatory subunit |
| YPR176c | BET2 | geranylgeranyltransferase type II beta subunit |
| YDR300c | PRO1 | glutamate 5-kinase |
| YPR035w | GLN1 | glutamate-ammonia ligase |
| YOR168w | GLN4 | glutaminyl-tRNA synthetase |
| YBR121c | GRS1 | glycine-tRNA ligase |
| YGR172c | YIP1 | golgi membrane protein |
| YDR302w | GPI11 | GPI11-protein involved in glycosylphosphatidylinositol (GPI) |
| | | biosynthesis |
| YGR267c | FOL2 | GTP cyclohydrolase I |
| YHR005c | GPA1 | GTP-binding protein alpha subunit of the pheromone pathway |
| YLR229c | CDC42 | GTP-binding protein of RAS superfamily |
| YPL218w | SAR1 | GTP-binding protein of the ARF family |
| YFL038c | YPT1 | GTP-binding protein of the rab family |
| YFL005w | SEC4 | GTP-binding protein of the ras superfamily |
| YLR293c | GSP1 | GTP-binding protein of the ras superfamily |
| YML064c | TEM1 | GTP-binding protein of the RAS superfamily |
| YPR165w | RHO1 | GTP-binding protein of the rho subfamily of ras-like proteins |
| YAL041w | CDC24 | GTP/GDP exchange factor for |
| CDC42P | YMR235c | RNA1 GTPase activating protein |
| YDR454c | GUK1 | guanylate kinase |
| YHR026w | PPA1 | H+-ATPase 23 KD subunit, vacuolar |
| YGL008c | PMA1 | H+-transporting P-type ATPase, major isoform, plasma membrane |
| YDR420w | HKR1 | Hansenula MraKII k9 killer toxin-resistance protein |
| YNL007c | SIS1 | heat shock protein |
| YOR232w | MGE1 | heat shock protein - chaperone |
| YLR259c | HSP60 | heat shock protein - chaperone, mitochondrial |
| YGL073w | HSF1 | heat shock transcription factor |
| YER125w | RSP5 | hect domain E3 ubiquitin-protein ligase |
| YMR290c | HAS1 | helicase associated with |
| SET1P | YPR033c | HTS1 histidine-tRNA ligase, mitochondrial |
| YOR244w | ESA1 | histone acetyltransferase |
| YJR139c | HOM6 | homoserine dehydrogenase |
| YBR153w | RIB7 | HTP reductase |
| YDR189w | SLY1 | hydrophilic suppressor of YPT1 and member of the SEC1P family |
| YDL139c | SCM3 | hypothetical protein |
| YDR016c | DAD1 | hypothetical protein |
| YDR396w | | hypothetical protein |
| YGL098w | | hypothetical protein |
| YGR128c | | hypothetical protein |
| YGR251w | | hypothetical protein |
| YHR083w | | hypothetical protein |
| YIR010w | | hypothetical protein |
| YJR023c | | hypothetical protein |
| YLR007w | | hypothetical protein |
| YLR033w | RSC58 | hypothetical protein |
| YLR112w | | hypothetical protein |
| YLR132c | | hypothetical protein |
| YLR145w | | hypothetical protein |
| YMR298w | | hypothetical protein |
| YNL150w | | hypothetical protein |
| YNL158w | | hypothetical protein |
| YNL258c | | hypothetical protein |
| YOL026c | | hypothetical protein |
| YPL012w | | hypothetical protein |
| YGL238w | CSE1 | importin-beta-like protein |
| YBR011c | IPP1 | inorganic pyrophosphatase, cytoplasmic |
| YML104c | MDM1 | intermediate filament protein |
| YDL058w | USO1 | intracellular protein transport protein |
| YLL011w | SOF1 | involved in 18S pre-rRNA production |
| YKL021c | MAK11 | involved in cell growth and replication of M1 dsRNA virus |
| YKR063c | LAS1 | involved in cell morphogenesis, cytoskeletal regulation and bud |
| | | formation |
| YGR099w | TEL2 | involved in controlling telomere length and position effect |
| YPL242c | IQG1 | involved in cytokinesis, has similarity to mammalian IQGAP |
| | | proteins |
| YJL090c | DPB11 | involved in DNA replication and S-phase checkpoint |
| YLR336c | SGD1 | involved in HOG pathway |
| YDR021w | FAL1 | involved in maturation of 18S rRNA |
| YGR158c | MTR3 | involved in mRNA transport |
| YJL050w | MTR4 | involved in nucleocytoplasmic transport of mRNA |
| YOR148c | SPP2 | involved in pre-mRNA processing |
| YLR197w | SIK1 | involved in pre-rRNA processing |
| YCL031c | RRP7 | involved in pre-rRNA processing and ribosome assembly |
| YBR257w | POP4 | involved in processing of tRNAs and rRNAs |
| YDR087c | RRP1 | involved in processing rRNA precursor species to mature rRNAs |
| YNL282w | POP3 | involved in processsing of tRNAs and rRNAs |
| YDR299w | BFR2 | involved in protein transport steps at the Brefeldin A blocks |
| YMR001c | CDC5 | involved in regulation of DNA replication |
| YNL251c | NRD1 | involved in regulation of nuclear pre-mRNA abundance |
| YIL046w | MET30 | involved in regulation of sulfur assimilation genes and cell cycle |
| | | progression |
| YGL201c | MCM6 | involved in replication |
| YGR095c | RRP46 | involved in rRNA processing |
| YDL153c | SAS10 | involved in silencing |
| YDR180w | SCC2 | involved in sister chromatid cohesion |
| YFR027w | ECO1 | involved in sister chromatid cohesion during replication |
| YGL120c | PRP43 | involved in spliceosome disassembly |
| YKR068c | BET3 | involved in targeting and fusion of ER to golgi transport vesicles |
| YML077w | BET5 | involved in targeting and fusion of ER to golgi transport vesicles |
| YDR082w | STN1 | involved in telomere length regulation |
| YPL117c | IDI1 | isopentenyl-diphosphate delta-isomerase |
| YNL189w | SRP1 | karyopherin-alpha or importin |
| YLR347c | KAP95 | karyopherin-beta |
| YGR140w | CBF2 | kinetochore protein complex CBF3, 110 KD subunit |
| YDR328c | SKP1 | kinetochore protein complex CBF3, subunit D |
| YMR168c | CEP3 | kinetochore protein complex, 71 KD subunit |
| YMR094w | CTF13 | kinetochore protein complex, CBF3, 58 KD subunit |
| YHR072w | ERG7 | lanosterol synthase |
| YPL160w | CDC60 | leucine-tRNA ligase, cytosolic |
| YDR037w | KRS1 | lysyl-tRNA synthetase, cytosolic |
| YDL055c | PSA1 | mannose-1-phosphate guanyltransferase |
| YER003c | PMI40 | mannose-6-phosphate isomerase |
| YGL065c | ALG2 | mannosyltransferase |
| YHR066w | SSF1 | mating protein |
| YKR008w | RSC4 | member of RSC complex, which remodels the structure of |
| | | chromatin |
| YPR019w | CDC54 | member of the CDC46P/MCM2P/MCM3P family |
| YBL023c | MCM2 | member of the MCM2P, MCM3P, CDC46P family |
| YMR208w | ERG12 | mevalonate kinase |
| YNR043w | MVD1 | mevalonate pyrophosphate decarboxylase |
| YDL126c | CDC48 | microsomal protein of CDC48/PAS1/SEC18 family of ATPases |
| YJL042w | MHP1 | microtubule-associated protein |
| YOR272w | YTM1 | microtubule-interacting protein |
| YGR029w | ERV1 | mitochondrial biogenesis and regulation of cell cycle |
| YJR045c | SSC1 | mitochondrial heat shock protein 70-related protein |
| YIL022w | TIM44 | mitochondrial inner membrane import receptor subunit |
| YJL143w | TIM17 | mitochondrial inner membrane import translocase subunit |
| YNR017w | MAS6 | mitochondrial inner membrane import translocase subunit |
| YNL131w | TOM22 | mitochondrial outer membrane import receptor complex subunit |
| YLR163c | MAS1 | mitochondrial processing peptidase |
| YDR376w | ARH1 | mitochondrial protein with similarity to human adrenodoxin |
| | | reductase and ferredoxin-NADP+ reductase |
| YDL003w | MCD1 | Mitotic Chromosome Determinant |
| YBL034c | STU1 | mitotic spindle protein |
| YBR156c | SLI15 | Mitotic spindle protein involved in chromosome segregation |
| YGL018c | JAC1 | molecular chaperone |
| YGR255c | COQ6 | monooxygenase |
| YBR236c | ABD1 | mRNA cap methyltransferase |
| YGL130w | CEG1 | mRNA guanylyltransferase (mRNA capping enzyme, alpha |
| | | subunit) |
| YER165w | PAB1 | mRNA polyadenylate-binding protein |
| YKL186c | MTR2 | mRNA transport protein |
| YPL085w | SEC16 | multidomain vesicle coat protein |
| YMR200w | ROT1 | mutant suppresses TOR2 mutation |
| YJL153c | INO1 | myo-inositol-1-phosphate synthase |
| YGL106w | MLC1 | MYO2P light chain |
| YOR326w | MYO2 | myosin heavy chain |
| YMR281w | GPI12 | N-acetylglucosaminyl phosphatidylinositol deacetylase |
| YPL076w | GPI2 | N-acetylglucosaminyl-phosphatidylinositol biosynthetic protein |
| YPL175w | SPT14 | N-acetylglucosaminyltransferase |
| YGR147c | NAT2 | N-acetyltransferase for N-terminal methionine |
| YLR195c | NMT1 | N-myristoyltransferase |
| YDR120c | TRM1 | N2,N2-dimethylguanine tRNA methyltransferase |
| YLR457c | NBP1 | NAP1P-binding protein |
| YDR464w | SPP41 | negative regulator of PRP3 and PRP4 gene expression |
| YPR168w | NUT2 | negative transcription regulator from artifical reporters |
| YER127w | LCP5 | NGG1P interacting protein |
| YLL036c | PRP19 | non-snRNP sliceosome component required for DNA repair |
| YHR170w | NMD3 | nonsense-mediated mRNA decay protein |
| YDL148c | NOP14 | nuclear and nucleolar protein with possible role in ribosome |
| | | biogenesis |
| YBL020w | RFT1 | nuclear division protein |
| YJR112w | NNF1 | nuclear envelope protein |
| YML031w | NDC1 | nuclear envelope protein |
| YGR218w | CRM1 | nuclear export factor, exportin |
| YJL034w | KAR2 | nuclear fusion protein |
| YPL124w | NIP29 | nuclear import protein |
| YGL122c | NAB2 | nuclear poly(A)-binding protein |
| YFR002w | NIC96 | nuclear pore protein |
| YGL092w | NUP145 | nuclear pore protein |
| YGL172w | NUP49 | nuclear pore protein |
| YGR119c | NUP57 | nuclear pore protein |
| YIL115c | NUP159 | nuclear pore protein |
| YJL041w | NSP1 | nuclear pore protein |
| YJL061w | NUP82 | nuclear pore protein |
| YCR093w | CDC39 | nuclear protein |
| YBR170c | NPL4 | nuclear protein localization factor and ER translocation component |
| YBR167c | POP7 | nuclear RNase P subunit |
| YER009w | NTF2 | nuclear transport factor |
| YAL025c | MAK16 | nuclear viral propagation protein |
| YDR432w | NPL3 | nucleolar protein |
| YNL061w | NOP2 | nucleolar protein |
| YPL043w | NOP4 | nucleolar protein |
| YOL144w | NOP8 | nucleolar protein required for 60S ribosome biogenesis |
| YDL208w | NHP2 | nucleolar rRNA processing protein |
| YHR072w-a | NOP10 | nucleolar rRNA processing protein |
| YHR089c | GAR1 | nucleolar rRNA processing protein |
| YJL039c | NUP192 | nucleoporin localize at the inner site of the nuclear membrane |
| YGL091c | NBP35 | nucleotide-binding protein |
| YEL002c | WBP1 | oligosaccharyl transferase beta subunit precursor |
| YGL022w | STT3 | oligosaccharyl transferase subunit |
| YMR149w | SWP1 | oligosaccharyltransferase delta subunit |
| YOR103c | OST2 | oligosaccharyltransferase epsilon subunit |
| YJL002c | OST1 | oligosaccharyltransferase, alpha subunit |
| YML065w | ORC1 | origin recognition complex, 104 KD subunit |
| YHR118c | ORC6 | origin recognition complex, 50 KD subunit |
| YNL261w | ORC5 | origin recognition complex, 50 kDa subunit |
| YPR162c | ORC4 | origin recognition complex, 56 KD subunit |
| YLL004w | ORC3 | origin recognition complex, 62 kDa subunit |
| YBR060c | ORC2 | origin recognition complex, 72 kDa subunit |
| YBR070c | SAT2 | osmotolerance protein |
| YCR057c | PWP2 | periodic tryptophan protein |
| YDR329c | PEX3 | peroxisomal assembly protein - peroxin |
| YLR060w | FRS1 | phenylalanyl-tRNA synthetase, alpha subunit, cytosolic |
| YKL203c | TOR2 | phosphatidylinositol 3-kinase |
| YNL267w | PIK1 | phosphatidylinositol 4-kinase |
| YMR079w | SEC14 | phosphatidylinositol(PI)/phosphatidylcholine(PC) transfer protein |
| YLR305c | STT4 | phosphatidylinositol-4-kinase |
| YDR208w | MSS4 | phosphatidylinositol-4-phosphate 5-kinase |
| YEL058w | PCM1 | phosphoacetylglucosamine mutase |
| YKL152c | GPM1 | phosphoglycerate mutase |
| YFL045c | SEC53 | phosphomannomutase |
| YMR220w | ERG8 | phosphomevalonate kinase |
| YDL235c | YPD1 | phosphorelay intermediate between SLN1P and SSK1P |
| YKR025w | RPC37 | Pol III transcription |
| YKR002w | PAP1 | poly(A) polymerase |
| YPL190c | NAB3 | polyadenylated RNA-binding protein |
| YNL317w | PFS2 | polyadenylation factor I subunit 2 required for mRNA 3′-end |
| | | processing, bridges two mRNA 3′-end processing factors |
| YJL025w | RRN7 | polymerase I specific transcription initiation factor |
| YLR430w | SEN1 | positive effector of tRNA-splicing endonuclease |
| YDR301w | CFT1 | pre-mRNA 3′-end processing factor CF II |
| YBR237w | PRP5 | pre-mRNA processing RNA-helicase |
| YAL032c | PRP45 | pre-mRNA splicing factor |
| YDL043c | PRP11 | pre-mRNA splicing factor |
| YJL203w | PRP21 | pre-mRNA splicing factor |
| YML046w | PRP39 | pre-mRNA splicing factor |
| YMR268c | PRP24 | pre-mRNA splicing factor |
| YDL030w | PRP9 | pre-mRNA splicing factor (snRNA-associated protein) |
| YDR088c | SLU7 | pre-mRNA splicing factor affecting 3′ splice site choice |
| YDR243c | PRP28 | pre-mRNA splicing factor RNA helicase of DEAD box family |
| YGR091w | PRP31 | pre-mRNA splicing protein |
| YLR223c | IFH1 | pre-rRNA processing machinery control protein |
| YAL043c | PTA1 | pre-tRNA processing protein/PF I subunit |
| YMR229c | RRP5 | processing of pre-ribosomal RNA |
| YHR024c | MAS2 | processing peptidase, catalytic 53 kDa (alpha) subunit, |
| | | mitochondrial |
| YJR017c | ESS1 | processing/termination factor 1 |
| YOR122c | PFY1 | profilin |
| YBR088c | POL30 | Proliferating Cell Nuclear Antigen (PCNA) |
| YPR137w | RRP9 | protein associated with the U3 small nucleolar RNA, required for |
| | | pre-ribosomal RNA processing |
| YNL221c | POP1 | protein component of ribonuclease P and ribonuclease MRP |
| YCL043c | PDI1 | protein disulfide-isomerase precursor |
| YKL019w | RAM2 | protein farnesyltransferase, alpha subunit |
| YLL031c | GPI13 | protein involved in glycosylphosphatidylinositol biosynthesis |
| YOL142w | RRP40 | protein involved in ribosomal RNA processing, component of the |
| | | exosome complex responsible for 3′ end processing and |
| | | degradation of many RNA species |
| YDL017w | CDC7 | protein kinase |
| YOR149c | SMP3 | protein kinase C pathway protein |
| YAR019c | CDC15 | protein kinase of the MAP kinase kinase kinase family |
| YPR107c | YTH1 | protein of the 3′ processing complex |
| YGL075c | MPS2 | Protein of the nuclear envelope/endoplasmic reticulum required for |
| | | spindle pole body assembly and normal chromosome segregation |
| YDR060w | MAK21 | protein required for 60S ribosomal subunit biogenesis |
| YOR372c | NDD1 | protein required for nuclear division |
| YER147c | SCC4 | protein required for sister chromatid cohesion |
| YML069w | POB3 | protein that binds to DNA polymerase I (PolI) |
| YDR164c | SEC1 | protein transport protein |
| YIL004c | BET1 | protein transport protein |
| YIL068c | SEC6 | protein transport protein |
| YLR208w | SEC13 | protein transport protein |
| YNL272c | SEC2 | protein transport protein |
| YPR055w | SEC8 | protein transport protein |
| YMR128w | ECM16 | putative DEAH-box RNA helicase |
| YDL098c | SNU23 | Putative RNA binding zinc finger protein |
| YDL031w | DBP10 | putative RNA helicase involved in ribosome biogenesis |
| YLR175w | CBF5 | putative rRNA pseuduridine synthase |
| YAL038w | CDC19 | pyruvate kinase |
| YBR190w | | questionable ORF |
| YDR053w | | questionable ORF |
| YDR355c | | questionable ORF |
| YDR412w | | questionable ORF |
| YGR190c | | questionable ORF |
| YKL036c | | questionable ORF |
| YKL083w | | questionable ORF |
| YLR076c | | questionable ORF |
| YLR101c | | questionable ORF |
| YLR140w | | questionable ORF |
| YLR198c | | questionable ORF |
| YLR317w | KRE34 | questionable ORF |
| YMR290w-a | | questionable ORF |
| YPL142c | | questionable ORF |
| YPL238c | | questionable ORF |
| YPL251w | | questionable ORF |
| YPR136c | FYV15 | questionable ORF |
| YDR002w | YRB1 | ran-specific GTPase-activating protein |
| YLR383w | RHC18 | recombination repair protein |
| YCL017c | NFS1 | regulates Iron-Sulfur cluster proteins, cellular Iron uptake, and Iron |
| | | distribution |
| YDR373w | FRQ1 | regulator of phosphatidylinositol-4-OH kinase protein |
| YOR294w | RRS1 | regulator of ribosome synthesis |
| YDR052c | DBF4 | regulatory subunit for CDC7P protein kinase |
| YKL193c | SDS22 | regulatory subunit for the mitotic function of type I protein |
| | | phosphatase |
| YEL032w | MCM3 | replication initiation protein |
| YMR213w | CEF1 | required during G2/M transition |
| YNL048w | ALG11 | required for asparagine-linked glycosylation |
| YLR088w | GAA1 | required for attachment of GPI anchor onto proteins |
| YIL106w | MOB1 | required for completion of mitosis and maintenance of ploidy |
| YPL211w | NIP7 | required for efficient 60S ribosome subunit biogenesis |
| YDL015c | TSC13 | required for elongation of the very long chain fatty acid (VLCFA) |
| | | moiety of sphingolipids |
| YGL145w | TIP20 | required for ER to Golgi transport |
| YDR166c | SEC5 | required for exocytosis |
| YLR166c | SEC10 | required for exocytosis |
| YGL142c | GPI10 | required for Glycosyl Phosphatdyl Inositol synthesis |
| YHR065c | RRP3 | required for maturation of the 35S primary transcript |
| YLR103c | CDC45 | required for minichromosome maintenance and initiation of |
| | | chromosomal DNA replication |
| YPR082c | DIB1 | required for mitosis |
| YKL089w | MIF2 | required for normal chromosome segregation and spindle integrity |
| YGR245c | SDA1 | required for normal organization of the actin cytoskeleton; required |
| | | for passage through Start |
| YGL033w | HOP2 | required for pairing of homologous chromosomes |
| YCL052c | PBN1 | required for post-translational processing of the protease B |
| | | precursor |
| PRB1P | YOR310c | NOP58 required for pre-18S rRNA processing |
| YKL172w | EBP2 | required for pre-rRNA processing and ribosomal subunit assembly |
| YHR062c | RPP1 | required for processing of tRNA and 35S rRNA |
| YAL033w | POP5 | required for processing of tRNAs and rRNAs |
| YBL018c | POP8 | required for processing of tRNAs and rRNAs |
| YGR030c | POP6 | required for processing of tRNAs and rRNAs |
| YML130c | ERO1 | required for protein disulfide bond formation in the ER |
| YMR005w | MPT1 | required for protein synthesis |
| YCL054w | SPB1 | required for ribosome synthesis, putative methylase |
| YIL150c | DNA43 | required for S-phase initiation or completion |
| YGR098c | ESP1 | required for sister chromatid separation |
| YJL074c | SMC3 | required for structural maintenance of chromosomes |
| YNL006w | LST8 | required for transport of permeases from the golgi to the plasma |
| | | membrane |
| YIR011c | STS1 | required for transport of RNA15P from the cytoplasm to the |
| | | nucleus |
| YML091c | RPM2 | ribonuclease P precursor, mitochondrial |
| YER070w | RNR1 | ribonucleoside-diphosphate reductase, large subunit |
| YJL026w | RNR2 | ribonucleoside-diphosphate reductase, small subunit |
| YGR180c | RNR4 | ribonucleotide reductase small subunit |
| YDR064w | RPS13 | ribosomal protein |
| YHL015w | RPS20 | ribosomal protein |
| YOL127w | RPL25 | ribosomal protein L23a.e |
| YPL143w | RPL33A | ribosomal protein L35a.e.c16 |
| YLR185w | RPL37A | ribosomal protein L37.e |
| YPR043w | RPL43A | ribosomal protein L37a.e |
| YNL178w | RPS3 | ribosomal protein S3.e |
| YML025c | YML6 | ribosomal protein, mitochondrial |
| YPL228w | CET1 | RNA 5′-triphosphatase (mRNA capping enzyme, beta subunit) |
| YDR381w | YRA1 | RNA annealing protein |
| YDR478w | SNM1 | RNA binding protein of RNase MRP |
| YDL207w | GLE1 | RNA export mediator |
| YOR046c | DBP5 | RNA helicase |
| YLL008w | DRS1 | RNA helicase of the DEAD box family |
| YNR038w | DBP6 | RNA helicase required for 60S ribosomal subunit assembly |
| YKL078w | DHR2 | RNA helicase, involved in ribosomal RNA maturation |
| YER172c | BRR2 | RNA helicase-related protein |
| YKL125w | RRN3 | RNA polymerase I specific transcription factor |
| YBL014c | RRN6 | RNA polymerase I specific transcription initiation factor |
| YML043c | RRN11 | RNA polymerase I specific transcription initiation factor |
| YHR058c | MED6 | RNA polymerase II transcriptional regulation mediator |
| YDR045c | RPC11 | RNA polymerase III subunit C11, required for RNA cleavage |
| | | activity and transcription termination |
| YPL007c | TFC8 | RNA Polymerase III transcription initiation factor TFIIIC (tau), 60 kDa |
| | | subunit |
| YKR086w | PRP16 | RNA-dependent ATPase |
| YIR015w | RPR2 | RNase P subunit |
| YPL266w | DIM1 | rRNA (adenine-N6,N6-)-dimethyltransferase |
| YCR035c | RRP43 | rRNA processing protein |
| YDL111c | RRP42 | rRNA processing protein |
| YDR280w | RRP45 | rRNA processing protein |
| YDR190c | RVB1 | RUVB-like protein |
| YPL235w | RVB2 | RUVB-like protein |
| YER043c | SAH1 | S-adenosyl-L-homocysteine hydrolase |
| YDR498c | SEC20 | secretory pathway protein |
| YLR078c | BOS1 | secretory pathway protein |
| YHR107c | CDC12 | septin |
| YNL038w | GPI15 | sequence and functional homologue of human Pig-H protein |
| YER133w | GLC7 | ser/thr phosphoprotein phosphatase 1, catalytic chain |
| YBL105c | PKC1 | ser/thr protein kinase |
| YPL209c | IPL1 | ser/thr protein kinase |
| YPR161c | SGV1 | ser/thr protein kinase |
| YHR102w | KIC1 | ser/thr protein kinase that interacts with CDC31P |
| YPL153c | RAD53 | ser/thr/tyr protein kinase |
| YDR062w | LCB2 | serine C-palmitoyltransferase subunit |
| YMR296c | LCB1 | serine C-palmitoyltransferase subunit |
| YHR205w | SCH9 | serine/threonine protein kinase involved in stress response and |
| | | nutrient-sensing signaling pathway |
| YDL028c | MPS1 | serine/threonine/tyrosine protein kinase |
| YDR023w | SES1 | seryl-tRNA synthetase, cytosolic |
| YLR066w | SPC3 | signal peptidase subunit |
| YPL210c | SRP72 | signal recognition particle protein |
| YPL243w | SRP68 | signal recognition particle protein |
| YIR022w | SEC11 | signal sequence processing protein |
| YLL003w | SFI1 | similarity to Xenopus laevis XCAP-C |
| YOR119c | RIO1 | similarity to a C. elegans ZK632.3 protein |
| YNL132w | KRE33 | similarity to A. ambisexualis antheridiol steroid receptor |
| YPL252c | YAH1 | similarity to adrenodoxin and ferrodoxin |
| YLR022c | | similarity to C. elegans and M. jannaschii hypothetical proteins |
| YDL060w | TSR1 | similarity to C. elegans hypothetical protein |
| YKL018w | SWD2 | similarity to C. elegans hypothetical protein |
| YKL059c | | similarity to C. elegans hypothetical protein |
| YNL313c | | similarity to C. elegans hypothetical protein |
| YPR133c | IWS1 | similarity to C. elegans hypothetical protein |
| YHR186c | | similarity to C. elegans hypothetical protein C10C5.6 |
| YKL095w | YJU2 | similarity to C. elegans hypothetical proteins |
| YKL088w | | similarity to C. tropicalis hal3 protein, to C-term. of SIS2P and to |
| | | hypothetical protein YOR054c |
| YBR159w | | similarity to human 17-beta-hydroxysteroid dehydrogenase |
| YLR051c | | similarity to human acidic 82 kDa protein |
| YDR013w | | similarity to human hypothetical KIAA0186 protein |
| YPL217c | BMS1 | similarity to human hypothetical protein KIAA0187 |
| YDR398w | | similarity to human KIAA0007 gene |
| YNL240c | NAR1 | similarity to human nuclear prelamin A recognition factor |
| YMR131c | RSA2 | similarity to human retinoblastoma-binding protein |
| YOL010w | RCL1 | similarity to human RNA 3′-terminal phosphate cyclase |
| YLR002c | | similarity to hypothetical C. elegans protein |
| YHR122w | | similarity to hypothetical C. elegans protein F45G2.a |
| YJL097w | | similarity to hypothetical C. elegans protein T15B7.2 |
| YHR188c | GPI16 | similarity to hypothetical C. elegans proteins F17c11.7 |
| YNL245c | | similarity to hypothetical protein CG2843 D. melanogaster |
| YDR361c | BCP1 | similarity to hypothetical protein S. pombe |
| YKL033w | | similarity to hypothetical protein S. pombe |
| YKL052c | ASK1 | similarity to hypothetical protein S. pombe |
| YDR367w | | similarity to hypothetical protein SPAC26H5.13c S. pombe |
| YKL014c | | similarity to hypothetical protein SPCC14G10.02 S. pombe |
| YPR105c | | similarity to hypothetical protein SPCC338.13 S. pombe |
| YDR066c | | similarity to hypothetical protein |
| YER139c | YHR036w | similarity to hypothetical protein |
| YGL247w | YHR088w | RPF1 similarity to hypothetical protein |
| YNL075w | YLR008c | similarity to hypothetical protein |
| YNL328c | YDL209c | similarity to hypothetical S. pombe protein |
| YGL047w | | similarity to hypothetical S. pombe protein |
| YNL124w | | similarity to hypothetical S. pombe protein |
| YLR106c | | similarity to Kaposi's sarcoma-associated herpes-like virus ORF73 |
| | | homolog gene |
| YOL133w | HRT1 | similarity to Lotus RING-finger protein |
| YPL093w | NOG1 | similarity to M. jannaschii GTP-binding protein |
| YNL207w | | similarity to M. jannaschii hypothetical protein MJ1073 |
| YGR211w | ZPR1 | similarity to M. musculus zinc finger protein |
| ZPR1 | YLL034c | similarity to mammalian valosin |
| YKL189w | HYM1 | similarity to mouse hypothetical calcium-binding protein and |
| | | D. melanogaster Mo25 gene |
| YOR077w | RTS2 | similarity to mouse KIN17 protein |
| YDL193w | | similarity to N. crassa hypothetical 32 kDa protein |
| YJL072c | | similarity to PIR: T40665 hypothetical protein SPBC725.13c S. pombe |
| YGR046w | | similarity to proline transport helper PTH1 C. albicans |
| YLR009w | | similarity to ribosomal protein L24.e.B |
| YPR112c | MRD1 | similarity to RNA-binding proteins |
| YJR067c | YAE1 | similarity to S. pombe SPAC2C4.12c putative phosphotransferase |
| YLL035w | GRC3 | similarity to S. pombe SPCC830.03 protein of unknown function |
| YNL308c | KRI1 | similarity to S. pombe and C. elegans hypothetical proteins |
| YDR434w | GPI17 | similarity to S. pombe hypothetical protein |
| YNL026w | | similarity to S. pombe hypothetical protein |
| YDR303c | RSC3 | similarity to transcriptional regulator proteins |
| YJL012c | VTC4 | similarity to YPL019c and YF1004w |
| YHR178w | STB5 | SIN3 binding protein |
| YNL263c | YIF1 | SLH1P Interacting Factor |
| YBL026w | LSM2 | Sm-like (Lsm) protein |
| YER112w | LSM4 | Sm-like (Lsm) protein |
| YLR438c-a | LSM3 | Sm-like (Lsm) protein |
| YKL196c | YKT6 | SNARE protein for Endoplasmic Reticulum-Golgi transport |
| YKL006c-a | SFT1 | SNARE-like protein |
| YGR074w | SMD1 | snRNA-associated protein |
| YFR005c | SAD1 | SnRNP assembly defective |
| YFL017w-a | SMX2 | snRNP G protein (the homologue of the human Sm-G) |
| YBR055c | PRP6 | snRNP(U4/U6)-associated splicing factor |
| YDR356w | NUF1 | spindle pole body component |
| YHR172w | SPC97 | spindle pole body component |
| YKL042w | SPC42 | spindle pole body component |
| YNL126w | SPC98 | spindle pole body component |
| YOR257w | CDC31 | spindle pole body component, centrin |
| YDR201w | SPC19 | spindle pole body protein |
| YER018c | SPC25 | spindle pole body protein |
| YKR037c | SPC34 | spindle pole body protein |
| YMR117c | SPC24 | spindle pole body protein |
| YOL069w | NUF2 | spindle pole body protein |
| YOR159c | SME1 | spliceosomal snRNA-associated Sm core protein required for |
| | | mRNA splicing, also likely associated with telomerase TLC1 RNA |
| YLR147c | SMD3 | spliceosomal snRNA-associated Sm core protein required for pre- |
| | | mRNA splicing |
| YJR022w | LSM8 | splicing factor |
| YKL012w | PRP40 | splicing factor |
| YKL165c | MCD4 | sporulation protein |
| YGR175c | ERG1 | squalene monooxygenase |
| YLR086w | SMC4 | Stable Maintenance of Chromosomes |
| YPL151c | PRP46 | strong similarity to A. thaliana PRL1 and PRL2 proteins |
| YJR072c | | strong similarity to C. elegans hypothetical protein and similarity to |
| | | YLR243w |
| YOL077c | BRX1 | strong similarity to C. elegans K12H4.3 protein |
| YLR397c | AFG2 | strong similarity to |
| CDC48 | YLR117c | CLF1 strong similarity to Drosophila putative cell cycle control |
| | | protein crn |
| YHR169w | DBP8 | strong similarity to DRS1P and other probable ATP-dependent |
| | | RNA helicases |
| YDR141c | DOP1 | strong similarity to Emericella nidulans developmental regulatory |
| | | gene, dopey (dopA) |
| YCL059c | KRR1 | strong similarity to fission yeast rev interacting protein mis3 |
| YNR053c | | strong similarity to human breast tumor associated autoantigen |
| YHR020w | | strong similarity to human glutamyl-prolyl-tRNA synthetase and |
| | | fruit fly multifunctional aminoacyl-tRNA synthetase |
| YDR091c | RLI1 | strong similarity to human RNase L inhibitor and M. jannaschii |
| | | ABC transporter protein |
| YOR145c | | strong similarity to hypothtical S. pombe protein and to |
| | | hypothetical C. elegans protein |
| YNL002c | RLP7 | strong similarity to mammalian ribosomal L7 proteins |
| YER036c | KRE30 | strong similarity to members of the ABC transporter family |
| YHR070w | TRM5 | strong similarity to N. crassa met-10+ protein |
| YIL003w | | strong similarity to NBP35P and human nucleotide-binding protein |
| YCR072c | | strong similarity to S. pombe trp-asp repeat containing protein |
| YLR186w | EMG1 | strong similarity to S. pombe hypothetical protein C18G6.07C |
| YAL047c | SPC72 | STU2P Interactant |
| YBL084c | CDC27 | subunit of anaphase-promoting complex (cyclosome) |
| YHR166c | CDC23 | subunit of anaphase-promoting complex (cyclosome) |
| YKL022c | CDC16 | subunit of anaphase-promoting complex (cyclosome) |
| YNL172w | APC1 | subunit of anaphase-promoting complex (cyclosome) |
| YLR272c | YCS4 | subunit of condensin protein complex |
| YDL008w | APC11 | subunit of the anaphase promoting complex |
| YDR118w | APC4 | subunit of the anaphase promoting complex |
| YKL013c | ARC19 | subunit of the ARP2/3 complex |
| YDL097c | RPN6 | subunit of the regulatory particle of the proteasome |
| YDL147w | RPN5 | subunit of the regulatory particle of the proteasome |
| YCR052w | RSC6 | subunit of the RSC complex |
| YFR037c | RSC8 | subunit of the RSC complex |
| YIL126w | STH1 | subunit of the RSC complex |
| YLR321c | SFH1 | subunit of the RSC complex |
| YBR091c | MRS5 | subunit of the TIM22-complex |
| YDL217c | TIM22 | subunit of the TIM22-complex |
| YHR005c-a | MRS11 | subunit of the TIM22-complex |
| YLR316c | TAD3 | subunit of tRNA-specific adenosine-34 deaminase |
| YIR012w | SQT1 | suppresses dominant-negative mutants of the ribosomal protein |
| | | QSR1 |
| YLR045c | STU2 | suppressor of a cs tubulin mutation |
| YOR329c | SCD5 | suppressor of clathrin deficiency |
| YNL222w | SSU72 | suppressor of cs mutant of SUA7 |
| YOR057w | SGT1 | suppressor of G2 allele of SKP1 |
| YLR026c | SED5 | syntaxin (T-SNARE) |
| YOR075w | UFE1 | syntaxin (T-SNARE) of the ER |
| YDR416w | SYF1 | synthetic lethal with |
| CDC40 | YJR064w | CCT5 T-complex protein 1, epsilon subunit |
| YML114c | TAF65 | TBP Associated Factor 65 KDa |
| YPL128c | TBF1 | telomere TTAGGG repeat-binding factor 1 |
| YKL058w | TOA2 | TFIIA subunit (transcription initiation factor), 13.5 kD |
| YOR194c | TOA1 | TFIIA subunit (transcription initiation factor), 32 kD |
| YPR086w | SUA7 | TFIIB subunit (transcription initiation factor), factor E |
| YBR198c | TAF90 | TFIID and SAGA subunit |
| YDR145w | TAF61 | TFIID and SAGA subunit |
| YDR167w | TAF25 | TFIID and SAGA subunit |
| YGL112c | TAF60 | TFIID and SAGA subunit |
| YMR236w | TAF17 | TFIID and SAGA subunit |
| YGR274c | TAF145 | TFIID subunit (TBP-associated factor), 145 kD |
| YML098w | TAF19 | TFIID subunit (TBP-associated factor), 19 kD |
| YML015c | TAF40 | TFIID subunit (TBP-associated factor), 40 KD |
| YMR227c | TAF67 | TFIID subunit (TBP-associated factor), 67 kD |
| YKR062w | TFA2 | TFIIE subunit (transcription initiation factor), 43 KD |
| YKL028w | TFA1 | TFIIE subunit (transcription initiation factor), 66 kD |
| YMR277w | FCP1 | TFIIF interacting component of CTD phosphatase |
| YGR186w | TFG1 | TFIIF subunit (transcription initiation factor), 105 kD |
| YGR005c | TFG2 | TFIIF subunit (transcription initiation factor), 54 kD |
| YDR311w | TFB1 | TFIIH subunit (transcription initiation factor), 75 kD |
| YPR025c | CCL1 | TFIIH subunit (transcription initiation factor), cyclin C component |
| YLR005w | SSL1 | TFIIH subunit (transcription initiation factor), factor B |
| YDR460w | TFB3 | TFIIH subunit (transcription/repair factor) |
| YPL122c | TFB2 | TFIIH subunit (transcription/repair factor) |
| YPR186c | PZF1 | TFIIIA (transcription initiation factor) |
| YGR246c | BRF1 | TFIIIB subunit, 70 kD |
| YNL039w | TFC5 | TFIIIB subunit, 90 kD |
| YGR047c | TFC4 | TFIIIC (transcription initiation factor) subunit, 131 kD |
| YAL001c | TFC3 | TFIIIC (transcription initiation factor) subunit, 138 kD |
| YOR110w | TFC7 | TFIIIC (transcription initiation factor) subunit, 55 kDa |
| YDR362c | TFC6 | TFIIIC (transcription initiation factor) subunit, 91 kD |
| YBR123c | TFC1 | TFIIIC (transcription initiation factor) subunit, 95 kD |
| YER148w | SPT15 | the TATA-binding protein TBP |
| YOR143c | THI80 | thiamin pyrophosphokinase |
| YIL078w | THS1 | threonyl tRNA synthetase, cytosolic |
| YOR074c | CDC21 | thymidylate synthase |
| YGR116w | SPT6 | transcription elongation protein |
| YML010w | SPT5 | transcription elongation protein |
| YBL093c | ROX3 | transcription factor |
| YBR049c | REB1 | transcription factor |
| YMR043w | MCM1 | transcription factor of the MADS box family |
| YOR174w | MED4 | transcription regulation mediator |
| YPL082c | MOT1 | transcriptional accessory protein |
| YBR193c | MED8 | transcriptional regulation mediator |
| YAL003w | EFB1 | translation elongation factor eEF1beta |
| YLR249w | YEF3 | translation elongation factor eEF3 |
| YNL163c | RIA1 | translation elongation factor eEF4 |
| YNL244c | SUI1 | translation initiation factor 3 (eIF3) |
| YPR016c | TIF6 | translation initiation factor 6 (eIF6) |
| YMR260c | TIF11 | translation initiation factor eIF1a |
| YPL237w | SUI3 | translation initiation factor eIF2 beta subunit |
| YER025w | GCD11 | translation initiation factor eIF2 gamma chain |
| YJR007w | SUI2 | translation initiation factor eIF2, alpha chain |
| YDR211w | GCD6 | translation initiation factor eIF2b epsilon, 81 kDa subunit |
| YLR291c | GCD7 | translation initiation factor eIF2b, 43 kDa subunit |
| YGR083c | GCD2 | translation initiation factor eIF2B, 71 kDa (delta) subunit |
| YOR260w | GCD1 | translation initiation factor eIF2bgamma subunit |
| YBR079c | RPG1 | translation initiation factor eIF3 (p110 subunit) |
| YDR429c | TIF35 | translation initiation factor eIF3 (p33 subunit) |
| YNL062c | GCD10 | translation initiation factor eIF3 RNA-binding subunit |
| YOR361c | PRT1 | translation initiation factor eIF3 subunit |
| YMR146c | TIF34 | translation initiation factor eIF3, p39 subunit |
| YOL139c | CDC33 | translation initiation factor eIF4E |
| YPR041w | TIF5 | translation initiation factor eIF5 |
| YBR143c | SUP45 | translational release factor |
| YJL125c | GCD14 | translational repressor of |
| GCN4 | YJL054w | TIM54 translocase for the insertion of proteins into the |
| | | mitochondrial inner membrane |
| YBL050w | SEC17 | transport vesicle fusion protein |
| YDR407c | TRS120 | TRAPP subunit of 120 kDa involved in targeting and fusion of ER |
| | | to golgi transport vesicles |
| YMR218c | TRS130 | TRAPP subunit of 130 kDa involved in targeting and fusion of ER |
| | | to golgi transport vesicles |
| YBR254c | TRS20 | TRAPP subunit of 20 kDa involved in targeting and fusion of ER |
| | | to golgi transport vesicles |
| YDR472w | TRS31 | TRAPP subunit of 31 kDa involved in targeting and fusion of ER |
| | | to golgi transport vesicles |
| YOL102c | TPT1 | tRNA 2′-phosphotransferase |
| YJL087c | TRL1 | tRNA ligase |
| YER168c | CCA1 | tRNA nucleotidyltransferase |
| YPL083c | SEN54 | tRNA splicing endonuclease alpha subunit |
| YLR105c | SEN2 | tRNA splicing endonuclease beta subunit |
| YMR059w | SEN15 | tRNA splicing endonuclease delta subunit |
| YAR008w | SEN34 | tRNA splicing endonuclease gamma subunit |
| YJL035c | TAD2 | tRNA-specific adenosine deaminase 2 |
| YLR136c | TIS11 | tRNA-specific adenosine deaminase 3 |
| YOL097c | WRS1 | tryptophan-tRNA ligase |
| YIL147c | SLN1 | two-component signal transducer |
| YGR185c | TYS1 | tyrosyl-tRNA synthetase |
| YDR240c | SNU56 | U1 snRNA protein, no counterpart in mammalian snRNP |
| YDR235w | PRP42 | U1 snRNP associated protein, required for pre-mRNA splicing |
| YMR240c | CUS1 | U2 snRNP protein |
| YPR178w | PRP4 | U4/U6 snRNP 52 KD protein |
| YHR165c | PRP8 | U5 snRNP protein, pre-mRNA splicing factor |
| YKL173w | SNU114 | U5 snRNP-specific protein |
| YGR048w | UFD1 | ubiquitin fusion degradation protein |
| YDR510w | SMT3 | ubiquitin-like protein |
| YPL020c | ULP1 | Ub1-specific protease |
| YBR243c | ALG7 | UDP-N-acetylglucosamine-1-phosphate transferase |
| YKL024c | URA6 | uridine-monophosphate kinase |
| YKL035w | UGP1 | UTP-glucose-1-phosphate uridylyltransferase |
| YMR197c | VTI1 | v-SNARE: involved in Golgi retrograde protein traffic |
| YGR094w | VAS1 | valyl-tRNA synthetase |
| YBR080c | SEC18 | vesicular-fusion protein, functional homolog of NSF |
| YMR049c | ERB1 | weak similarity to A. thaliana PRL1 protein |
| YOR353c | | weak similarity to adenylate cyclases |
| YLR064w | | weak similarity to Anopheles NADH-ubiquinone oxidoreductase, |
| | | chain 4 |
| YLR010c | | weak similarity to Aquifex aeolicus adenylosuccinate synthetase |
| YHR074w | QNS1 | weak similarity to B. subtilis spore outgrowth factor B |
| YMR211w | | weak similarity to beta tubulins |
| YJL010c | | weak similarity to C. elegans hypothetical protei ZK792.5 |
| YJL104w | MIA1 | weak similarity to C. elegans hypothetical protein F45G2.c |
| YGR280c | | weak similarity to CBF5P |
| YJL011c | | weak similarity to chicken hypothetical protein |
| YHR085w | | weak similarity to fruit fly brahma transcriptional activator |
| YNL110c | | weak similarity to fruit fly RNA-binding protein |
| YPL126w | NAN1 | weak similarity to fruit fly TFIID subunit p85 |
| YHR040w | | weak similarity to HIT1P |
| YJR136c | | weak similarity to human 3′,5′-cyclic-GMP phosphodiesterase |
| YJL091c | | weak similarity to human G protein-coupled receptor |
| YOR056c | | weak similarity to human phosphorylation regulatory protein HP- |
| | | 10 |
| YLL037w | | weak similarity to human platelet-activating factor receptor |
| YGL108c | | weak similarity to hypotetical S. pombe protein |
| YJR012c | | weak similarity to hypothetical protein B17C10.80 N. crassa |
| YNL260c | | weak similarity to hypothetical protein S. pombe |
| YPL233w | | weak similarity to hypothetical protein S. pombe |
| YJR041c | | weak similarity to hypothetical protein SPAC2G11.02 S. pombe |
| YJR141w | | weak similarity to hypothetical protein SPBC1734.10c S. pombe |
| YOR004w | | weak similarity to hypothetical protein |
| YDR339c | YBR168w | weak similarity to hypothetical protein |
| YLR324w | YDR339c | weak similarity to hypothetical protein |
| YOR004w | YDR413c | weak similarity to NADH dehydrogenase |
| YHR052w | | weak similarity to P. yoelii rhoptry protein |
| YBL004w | | weak similarity to Papaya ringspot virus polyprotein |
| YOR281c | PLP2 | weak similarity to phosducins |
| YGR156w | PTI1 | weak similarity to PIR: A40220 cleavage stimulation factor 64 K |
| | | chain - human |
| YHR197w | | weak similarity to PIR: T22172 hypothetical protein F44E5.2 C. elegans |
| YGR198w | | weak similarity to PIR: T38996 hypothetical protein SPAC637.04 |
| | | S. pombe |
| YLR143w | | weak similarity to Pyrococcus horikoshii hypothetical protein |
| | | PHBJ017 |
| YDL166c | | weak similarity to Pyrococcus horikoshii hypothetical protein |
| | | PHBJ019 |
| YNL182c | | weak similarity to S. pombe hypothetical protein |
| YDR365c | | weak similarity to Streptococcus M protein YBR155w |
| CNS1 | | weak similarity to stress-induced STI1P |
| YCR054c | CTR86 | weak similarity to THR4P |
| YJR046w | TAH11 | weak similarity to Xenopus vimentin 4 |
| YHR196w | | weak similarity to YDR398w |
| YGL113w | SLD3 | weak similarity to YOR165w |

G. Yeast Codon Bias

To obtain optimal expression of a heterologous peptidase in yeast cells, nucleic acids encoding peptidases will be designed and synthesized according to yeast codon preference. For example, the inventors have synthesized the gene that encodes light-chain of BoNT/B. Clostridial DNA contains a high content of adenine and thymine, which can terminate transcription in yeast. Without changing the amino acid sequence of the light-chain, the construct eliminates A/T rich stretches and rare yeast condons. The resulting peptidase encoded by the synthetic gene efficiently cleaves the recombinant substrate in yeast cells, causing cell death. The following table, derived from Bennetzen & Hall (1982), lists yeast codon preferences.

TABLE 4
YEAST CODON PREFERENCES

| | |
|---|---|
| Codon | Preferred Triplet |

| | |
|---|---|
| Ala | GCU, GCC |
| Ser | UCU, UCC |
| Thr | ACU, ACC |
| Val | GUU, GUC |
| Ile | AUU, AUC |
| Asp | GAC |
| Phe | UUC |
| Tyr | UAC |
| Cys | UGU |
| Asn | AAC |
| His | CAC |
| Arg | AGA |
| Glu | GAA |
| Leu | UUG |
| Lys | AAG |
| Gly | GGU |
| Gln | CAA |
| Pro | CCA |
| Met | AUG |
| Trp | UGG |

V. Assays

As discussed above, the yeast cell system of the present invention is designed such that cleavage of the recombinant protein substrate by the heterologous peptidase causes cell death. Conditional (or regulated) expression of the heterologous peptidase permits growth of the yeast host cell without cell death. Inclusion of an inhibitor of the heterologous peptidase, under conditions supporting peptidase expression, aborts the enzymatic activity of the peptidase and permits proliferation of the yeast cells. One can introduce a large number of biological peptides, proteins, small molecules, or intracellular recombinant antibodies in yeast cells bearing the heterologous peptidase and the recombinant protein substrate to directly and rapidly select/identify specific peptidase inhibitors that permit yeast cell growth.

A. Genetic Selection

A DNA library encoding potential protein inhibitors can be transformed in yeast cells with high frequency (at 10⁴-10⁵ transformants/microgram plasmid DNA). Transformants are plated on agar plates containing an inducer of the peptidase gene, and an amino acid drop-out for the selection of plasmid marker. Most yeast transformants are not able to grow on galactose containing plates since the heterologous peptidase is expressed, and those not transformed will additionally not grow because of the absence of the plasmid. However, the presence of a plasmid-borne peptidase inhibitor in a yeast transformant will lead to cell growth and formation of a colony. The plasmid DNA can be recovered using standard DNA purification procedure, and the DNA sequence of the inhibitor can be determined through DNA sequencing, if not previously known.

B. High-Throughput Screen (HTS)

Small molecule peptide and chemical inhibitors can be identified by using clear bottom multi-well plates (currently, 96- and 384-well plates are commercially available). Yeast cells are diluted and distributed equally in each well in the presence of yeast growth media containing galactose. Compounds are distributed to each well and yeast cell growth is monitored by visual inspection or measured with a multi-well plate reader (at A₆₀₀). The presence of a toxin inhibitor will lead to yeast cell growth and increased turbidity in a well. This HTS assay is a standard practice and has been successfully employed in the identification of small molecule inhibitors of process distinct from ours (see Hughes, 2002). To overcome the potentially limiting factor of cell penetration, one can enhance cell permeability of the yeast cells by treating with specific chemicals such as polymixin B (Boguslaski, 1985), or using yeast strain that carries a cell wall mutation (Brendel, 1976). Also contemplated are yeast cells that are impaired in multidrug efflux (Wolfger et al., 2001).

### VI. EXAMPLES

The following examples are included to further illustrate various aspects of the invention. It should be appreciated by those of skill in the art that the techniques disclosed in the examples which follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

A method is presented which allows one to directly select for intracellular inhibitors of the light chain (LC) peptidase of botulinum neurotoxin (serotype B) BoNTB and other bacterial toxins. A yeast mutant that would be susceptible to the lethal effects of intracellular BoNTB/LC was generated. This toxin is an endopeptidase that cleaves a specific QF peptide bond in synaptobrevin (Sb), a neuronal cell protein that is required for vesicle fusion to the presynaptic membrane. Yeast (Saccharomyces cerevisiae) possess two functionally redundant Sb homologs, Snc1 and Snc2, that are essential for secretory vesicle fusion to the plasma membrane. Snc1/2 are structurally and functionally related to Sb; however Snc1/2 lack the QF sequence that is recognized by BoNTB/LC. Therefore, whether a Snc2 protein that contains a portion of Sb (with the QF sequence) could be rendered inactive by the expression of BoNTB/LC in yeast cells was investigated.

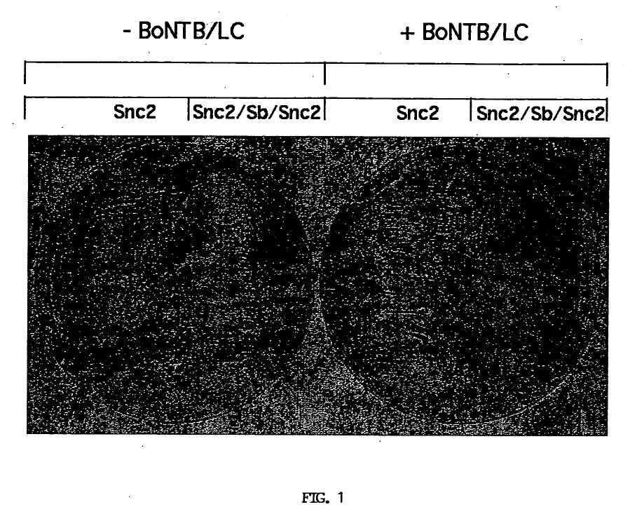

Two yeast strains that lack Snc1 were constructed. Growth of the first mutant is dependent on expression of Snc2. Growth of the second Δsnc1 mutant is dependent on the expression of a Snc2/Sb/Snc2 fusion. As was shown, both of these strains can grow when BoNTB/LC expression is repressed by the regulatable GAL1 promoter. The left-hand side of FIG. 1 depicts an agar plate containing glucose. In contrast, derepression of the GAL1 promoter was lethal to cells that expressed the Snc2/Sb/Snc2 fusion, which were grown in an agar plate containing glucose, but not to cells that expressed the Snc2 protein, which were grown in an agar plate containing galactose and shown in the right-had side of FIG. 1.

This yeast cell based assay provides a powerful tool with which to directly select for intracellular inhibitors of BoNTBLC. Yeast expression libraries of scFv (single chain fragment variable) antibodies may be introduced into yeast that contain the Snc2/Sb/Snc2 fusion, selecting for growth in the presence of galactose.

### Example 2

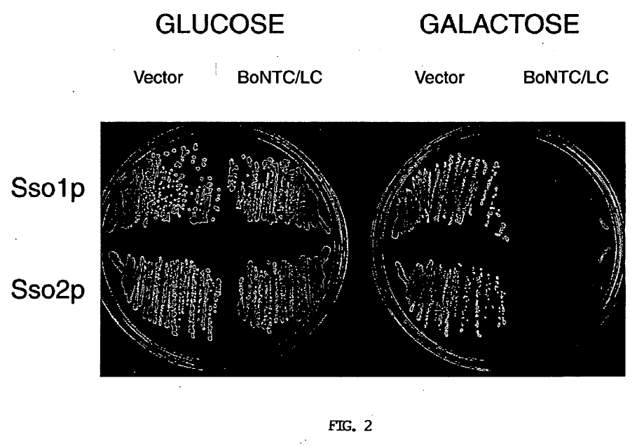

In a second embodiment, the inventors synthesized the gene corresponding to BoNTC/LC, eliminating A/Trich stretches without changing the amino acid sequence. The gene was then placed under control of the GAL1 promotor, which can be regulated in yeast: ON in the presence of galactose and OFF in the presence of glucose. The GAL1-BoNTC/LC construct and a control plasmid vector lacking GAL1-BoNTC/LC were then introduced into yeast cells that expressed either Sso1p or Sso2p. As shown in FIG. 2, the vector control and GAL1-BoNTC/LC were not lethal to yeast cells that were grown in the presence of glucose (left hand dish). In addition, the vector did not interfere with cell growth in the presence of galactose (right hand dish). Importantly, the GAL1-BoNTC/LC construct strongly inhibited cell growth in the presence of galactose (right hand panel). These data demonstrate that BoNTC/LC exerts a severe growth defect on yeast that express either Sso1p or Sso2p.

A clue to the substrate specificity of BoNTC/LC may already exist. A careful examination of FIG. 2 (right hand dish) shows the Sso1p strain grows slightly better than the Sso2p strain in the presence of BoNTC/LC. One explanation for this result could be that BoNTC/LC cleaves Sso2p somewhat better than Sso1p. In support of this idea, Sso2p exhibits slightly stronger similarity to syntaxin at the cleavage site as compared to Sso1p (FIG. 3). In particular, syntaxin, Sso1p, and Sso2p have Lys, Asp, Asn, respectively, at the P2 position. These differences result in a change in amino acid charge from positive to negative for Sso1p, but only a change of positive to polar for Sso2p.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods, and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the scope of the invention as defined by the appended claims.

### VII. REFERENCES

The following references, to the extent that they provide exemplary procedural or other details supplementary to those set forth herein, are specifically incorporated herein by reference:
U.S. Pat. No. 5,077,204
U.S. Pat. No. 5,384,253
EP 73,675
Ahkong et al., Nature, 253:194-195, 1975a.
Almendro et al., J. Immunol., 157(12):5411-5421, 1996.
Angel et al., Cell, 49:729, 1987b.
Angel et al., Mol. Cell. Biol., 7:2256, 1987a.
Anné and Peberdy, Archives of Microbiol., 105:201-205, 1975.
Bennetzen and Hall, J. Biol. Chem., 257(6):3026-302631, 1982.
Blanar et al., EMBO J, 8:1139, 1989.
Boguslaski, Contemp. Issues Clin. Biochem., 1985;2:63-88, 1985.
Chandler et al., Cell, 33:489, 1983.
Chatterjee et al., Proc. Natl. Acad. Sci. USA, 86:9114, 1989.
Gumpert, Archives of Microbiol., 126:263-269, 1980.
Gupthar, J. Canadian Microbiol., 38:1233-1237, 1992.
Halfmann et al., Current Genetics, 6:25-28, 1982.
Haslinger and Karin, Proc. Natl. Acad. Sci. USA, 82:8572, 1985.
Hensel et al., Lymphokine Res., 8:347, 1989.
Huang et al., Cell, 27:245, 1981.
Hug et al., Mol. Cell. Biol., 8:3065, 1988.
Imagawa et al., Cell, 51:251, 1987.
Imperiale and Nevins, Mol. Cell. Biol., 4:875, 1984.
Janderová et al., J. Basic Microbiol., 30:499-505, 1990.
Kao and Michayluk, Planta, 115:355-367, 1974.
Karin et al., Mol. Cell. Biol., 7:606, 1987.
Klinner and Böttcher, Zeitschrift für allgemeine Mikrobiologie, 24:539-544, 1984.
Kraus et al. FEBS Lett., 428(3):165-170, 1998.
Kunz et al., Nucl. Acids Res., 17:1121, 1989.
Lareyre et al., J. Biol. Chem., 274(12):8282-8290, 1999.
Lee et al., Biochem. Biophys. Res. Commun., 240(2):309-313, 1997.
Lee et al., Nature, 294:228, 1981.
Lee et al., Nucleic Acids Res., 12:4191-206, 1984.
Majors and Varmus, Proc. Natl. Acad. Sci. USA, 80:5866, 1983.
McNeall et al., Gene, 76:81, 1989.
Molnar and Sipiczki, Current Genetics, 24:45-52, 1993.
Mordacq and Linzer, Genes and Dev., 3:760, 1989.
Nomoto et al., Gene, 236(2):259-271, 1999.
Okazaki et al., Nucl. Acids Res., 18:6485-6489, 1990.
Palmiter et al., Nature, 300:611, 1982.
Perez et al., Current Genetics, 8:575-580, 1984.
Pina et al., Applied Environ. Microbiol., 51:995-1003, 1986.
Ponta et al., Proc. Natl. Acad. Sci. USA, 82:1020, 1985.
Provost et al., FEMS Microbiol. Lett., 3:309-312, 1978.
Resendez Jr. et al., Mol. Cell. Biol., 8:4579, 1988.
Rittling et al., Nuc. Acids Res., 17:1619, 1989.
Sakai et al., Genes and Dev., 2:1144, 1988.
Sarachek and Rhoads, Current Genetics, 4:221-222, 1981.
Searle et al., Mol. Cell. Biol., 5:1480, 1985.
Skala et al., Current Genetics, 13:101-104, 1988.
Spencer et al., Current Genetics, 9:649-652, 1985.
Stuart et al., Nature, 317:828, 1985.
Suga and Hatakeyama, Yeast, 18:1015-1021, 2001.
Svoboda, Acta Histochemica, 23:211-217, 1981.
Svoboda, Archives of Microbiol., 110:313-318, 1976.
Svoboda, J. General Microbiol., 109:169-175,1978.
Tavernier et al., Nature, 301:634, 1983.
Taylor and Kingston, Mol. Cell. Biol., 10:165, 1990a.
Taylor and Kingston, Mol. Cell. Biol., 10:176, 1990b.
Taylor et al., J. Biol. Chem., 264:15160, 1989.
Tsumaki et al., J. Biol. Chem., 273(36):22861-22864, 1998.
van Solingen and van der Plaat, J. Bacteriology, 130:946-947, 1977.
Weber et al., Current Genetics, 4:165-166, 1981.
Wilson et al., Molecular Gen. Genetics, 186:95-100, 1982.
Wolfger et al., Res. Microbiol., 152:375-389, 2001.
Wu et al., Biochem. Biophys. Res. Commun., 233(1):221-6, 1997.

### Zhao-Emonet et al., Biochim. Biophys. Acta, 1442(2-3):109-119, 1998.

1. A method of identifying an endopeptidase inhibitor comprising:
(a) providing a yeast cell, wherein said cell expresses a polypeptide that is essential to yeast cell viability or growth, wherein said polypeptide comprises or has been modified to comprise a cleavage site for said endopeptidase;
(b) contacting said yeast cell and said endopeptidase in the presence of a candidate substance; and
(c) assessing the viability and/or growth of said yeast cell,
wherein improved viability and/or growth of said yeast cell in the presence of said candidate substance, as compared to viability and/or growth of said yeast cell in the absence of said candidate substance, identifies said candidate substance as a endopeptidase inhibitor.

2. The method of claim 1, wherein said endopeptidase is a serine endopeptidase, and cysteine endopeptidase, an aspartic endopeptidase or a metallo endopeptidase.

3. The method of claim 1, wherein said endopeptidase is a bacterial toxin endopeptidase.

4. The method of claim 3, wherein said toxin endopeptidase is Botulinum neurotoxin, and said endopeptidase cleavage site is Q/F or K/A.

5. The method of claim 1, wherein said essential polypeptide is Snc1, Snc2, Sso1 or Sso2.

6. The method of claim 1, wherein yeast cell viability is measured.

7. The method of claim 6, wherein yeast cell viability is measured by standard culture methods, by flow cytometry by selective staining, by the slide viability method, by flocculation test, or by fermentation test.

8. The method of claim 1, wherein yeast cell growth is measured.

9. The method of claim 8, wherein yeast cell growth is measured by measuring incorporation of radioactive nucleotides or by cell counting.

10. The method of claim 1, wherein said yeast cell further comprises a null mutation in a functionally redundant homolog of said essential polypeptide.

11. The method of claim 1, wherein said yeast cell further comprises an endopeptidase transgene under the control of an inducible promoter, and contacting comprises growing said yeast cell under conditions that induce said promoter, thereby permitting expression of said endopeptidase in said yeast cell.

12. The method of claim 11, wherein said inducible promoter is a yeast inducible promoter.

13. The method of claim 12, wherein said yeast inducible promoter is GAL1 or GAL10, and said conditions that induce said promoter comprises culturing said yeast in galactose.

14. The method of claim 11, wherein said inducible promoter is a non-yeast inducible promoter.

15. The method of claim 14, wherein said non-yeast inducible promoter is a tetracycline-responsive promoter.

16. The method of claim 1, wherein the candidate substance is a peptide or polypeptide and providing said peptide or polypeptide comprises contacting said yeast cell with an expression construct encoding said peptide or polypeptide.

17. The method of claim 16, wherein said polypeptide is an antibody or an enzyme.

18. The method of claim 1, wherein said candidate substance is a peptide.

19. The method of claim 1, wherein said candidate substance is an organopharmaceutical.

20. The method of claim 1, wherein said candidate substance is a siRNA.

21. A yeast cell that expresses a polypeptide that is essential to yeast cell viability or growth, wherein said polypeptide comprises a heterologous cleavage site for a endopeptidase.

22. The yeast cell of claim 21, further comprising a transgene encoding said endopeptidase under the control of an inducible promoter.

23. The yeast cell of claim 22, wherein said inducible promoter is a yeast inducible promoter.

24. The yeast cell of claim 22, wherein said inducible promoter is a non-yeast inducible promoter.

25. The yeast cell of claim 21, wherein said yeast cell further comprises a mutation a functionally redundant homolog of said polypeptide that comprises said heterologous cleavage site.
